(19) United States
(12) Patent Application Publication
(10) Pub. No.: US 2024/0382613 A1
(43) Pub. Date: Nov. 21, 2024

(54) METHODS AND SYSTEMS FOR ULTRASOUND STIMULATED STRUCTURES AND DELIVERY OF THERAPEUTIC SPECIES

(71) Applicant: The Penn State Research Foundation, University Park, PA (US)

(72) Inventors: Julien H. Arrizabalaga, State College, PA (US); Mohammad Abu-Laban, Bethesda, MD (US); Julianna C. Simon, State College, PA (US); Daniel J. Hayes, State College, PA (US); Ferdousi Sabera Rawnaque, University Park, PA (US); Tyus Yeingst, University Park, PA (US)

(21) Appl. No.: 18/689,620

(22) PCT Filed: Sep. 7, 2022
(86) PCT No.: PCT/US2022/042708

§ 371 Date: Mar. 6, 2024

Related U.S. Application Data
(60) Provisional application No. 63/241,183, filed on Sep. 7, 2021.
(60) Provisional application No. 63/338,799, filed on May 5, 2022.

(51) Int. Cl. A61K 47/69; A61K 41/00; A61K 47/60; A61P 19/00

(52) U.S. Cl. CPC A61K 47/6903; A61K 41/0028; A61K 47/60; A61P 19/00

(57) ABSTRACT

An exemplary embodiment of the present disclosure provides compositions and methods of using compositions. A composition described herein comprises a hydrogel, a therapeutic species, and a linker joining the hydrogel to the therapeutic species. The linker joining the hydrogel to the therapeutic species comprises a Diels-Alder cyclo-addition reaction product. Some aspects of this disclosure relate to methods of delivering a therapeutic species to a subject. The method comprises disposing the composition in the subject and initiating a retro Diels-Alder reaction to decompose the Diels-Alder cyclo-addition product, thereby severing the linker and decoupling the therapeutic species from the hydrogel.

### CROSS-REFERENCE TO RELATED APPLICATIONS

This application claims the benefit of U.S. Provisional Application Ser. No. 63/241,183, filed on 7 Sep. 2021, and U.S. Provisional Application Ser. No. 63/338,799, filed on 5 May 2022, both of which is incorporated herein by reference in their entirety as if fully set forth below.

### FEDERALLY SPONSORED RESEARCH STATEMENT

This invention was made with government support under Grant No. W81XWH-21-1-0052 awarded by the United States Army/MRMC. The Government has certain rights in the invention.

### TECHNICAL FIELD

The various embodiments of the present disclosure relate generally to structures for controlled release of therapeutic species, and more particularly to hydrogels that undergo retro Diels-Alder cleavage reaction under focused ultrasound (fUS) stimulation.

### BACKGROUND

Implantable structures and drug delivery systems that allow for controlled release of drugs can provide improved outcomes in repairing bone and tissue that are traditionally difficult to access after surgical reconstruction or during regenerative medicine. When delivering therapeutics directly to the target site, conventional polymer-based systems often rely on passive diffusion and polymer degradation but lack temporal control of therapeutic release. Similarly, polymeric implantable structures for bone regeneration suffer from uncontrolled polymer degradation and lack of structure. Some internal stimulation such as chemical triggers can assist in controlling temporal release, but often require a specific immuno-response in the subject or an additional chemical to be incorporated into the drug delivery system to initiate polymer degradation to release the therapeutic. External physical stimulation such as light, heat, or magnetic field can also initiate polymer degradation and therapeutic release, but these methods expose the entire region to the physical stimulation, which can lead to off-target ionizing or burning. Additionally, such stimuli fail to reach deep tissue polymer-based drug delivery systems and therefore lack control of intensity or frequency of drug release.

Therefore, compositions that can readily degrade to release therapeutics to a target under a potent and safe external stimulus may provide a beneficial drug delivery system and limit off-target ionizing or burning to deep tissue targets. In the disclosure herein, a polymer-based drug delivery system made up of a crosslinked 3D network with Diels-Alder linkers undergoes degradation and release of therapeutics when stimulated with focused ultrasound to limit off-target effects while controlling the rate of therapeutic release.

### SUMMARY

The subject of this disclosure includes methods of delivering or uses of compositions having hydrogels that undergo retro Diels-Alder cleavage reaction under focused ultrasound stimulation to optionally release therapeutic species to a target tissue. An exemplary embodiment of the present disclosure provides a composition including a hydrogel and a therapeutic species coupled to the hydrogel. The hydrogel can include a linker having a Diels-Alder cyclo-addition reaction product. The linker can be configured to undergo a reversible retrograde cleavage reaction to release the therapeutic species from the hydrogel upon exposure to a triggering event.

In some embodiments, the therapeutic species can be encapsulated in the hydrogel.

In some embodiments, the triggering event can include pulsed waves of acoustic energy.

In some embodiments, the pulsed waves of acoustic energy can comprise a waveform, a pulse duration, and a pulse repetition frequency.

In some embodiments, the waveform can comprise a positive peak pressure amplitude ranging from about 40 megapascals (MPa) to about 100 MPa and a negative peak pressure amplitude ranging from about 10 MPa to about 30 MPa. The pulse duration can include a number of cycles ranging from about 1 cycle to about 20,000 cycles within a timeframe ranging from about 1 microseconds (μs) to about 20 milliseconds (ms). The pulse repetition frequency can include a frequency ranging from about 0.1 hertz (Hz) to about 100 Hz. A period of treatment time can range from about 30 seconds to about 300 seconds.

In some embodiments, the hydrogel can include a biocompatible polymer. The biocompatible polymer can include substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxy butyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

In some embodiments, the therapeutic species can include at least one of a small molecule, a nucleic acid, a peptide, a protein, a microRNA mimetic, and combinations thereof. In some embodiments, the linker can include a reaction product of a diene and a dienophile. The diene can include at least one of a substituted or unsubstituted furan, thiophene, or pyrrole. The dienophile can include at least one of a substituted or unsubstituted alkene or alkyne.

An exemplary embodiment of the present disclosure provides a composition comprising a first material and a first therapeutic species. The first material can include a first precursor. The first therapeutic species can include a second precursor. The first and second precursor can form a Diels-Alder cyclo-addition reaction product. The Diels-Alder cyclo-addition reaction product can be configured to undergo a retro-Diels-Alder reaction upon exposure to a first triggering event to release the first therapeutic species.

In some embodiments, the composition can further comprise a second material and a second therapeutic species. The second material can include a third precursor. The second therapeutic species can include a fourth precursor. The third and fourth precursor can form a Diels-Alder cyclo-addition reaction product. In some embodiments, the third precursor and fourth precursor can be different than the first precursor and the second precursor.

In some embodiments, the third and fourth precursors can be configured to undergo a retro-Diels-Alder reaction upon exposure to a second triggering event to release the second therapeutic species.

In some embodiments, the first triggering event can be different from the second triggering event, such that the first therapeutic species and second therapeutic species are released at different triggering events.

In some embodiments, at least one of the first triggering event or the second triggering event can include pulsed waves of acoustic energy.

In some embodiments, the pulsed waves of acoustic energy can include a waveform, a pulse duration, and a pulse repetition frequency.

In some embodiments, when the first triggering event and the second triggering event comprise pulsed waves of acoustic energy, the second triggering event can include pulsed waves of acoustic energy comprising at least one of the waveform, pulse duration, or pulse repetition frequency different from the first triggering event.

In some embodiments the waveform can comprise a positive peak pressure amplitude ranging from about 40 megapascals (MPa) to about 100 MPa and a negative peak pressure amplitude ranging from about 10 MPa to about 30 MPa. The pulse duration can comprise a number of cycles ranging from about 1 cycle to about 20,000 cycles within a timeframe ranging from about 1 microseconds (μs) to about 20 milliseconds (ms). The pulse repetition frequency can comprise a frequency ranging from about 0.1 hertz (Hz) to about 100 Hz. A period of treatment time can range from about 30 seconds to about 300 seconds.

In some embodiments, the first material can include a biocompatible polymer. The second material can include a biocompatible polymer. The biocompatible polymer can comprise substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxy butyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

In some embodiments, the first therapeutic species can include at least one of a small molecule, a nucleic acid, a peptide, a protein, a microRNA mimetic, and combinations thereof.

In some embodiments, the first precursor can include a diene selected from a substituted or unsubstituted furan, thiophene, or pyrrole. The second precursor can include a dienophile comprising a substituted or unsubstituted alkene or alkyne. The third precursor can include a diene selected from a substituted or unsubstituted furan, thiophene, or pyrrole. The fourth precursor can include a dienophile comprising a substituted or unsubstituted alkene or alkyne.

An exemplary embodiment of the present disclosure provides a method of delivering a therapeutic species to a subject. The method can include disposing a composition comprising a hydrogel and a therapeutic species in the subject and exposing the composition to pulsed waves of acoustic energy. The hydrogel can include a linker joining the hydrogel to the therapeutic species. Exposing the composition to pulsed waves of acoustic energy can initiate a reversible retrograde cleavage reaction to severe the linker and decouple the therapeutic species from the hydrogel.

In some embodiments, the method can further comprise encapsulating the therapeutic species within the hydrogel. In some embodiments, the method can further comprise coupling the therapeutic species with the hydrogel via a Diels-Alder reaction product comprising a first precursor on the hydrogel and a second precursor on the therapeutic species.

In some embodiments, the pulsed waves of acoustic energy can include a waveform, a pulse duration, and a pulse repetition frequency.

In some embodiments the waveform can comprise a positive peak pressure amplitude ranging from about 40 megapascals (MPa) to about 100 MPa and a negative peak pressure amplitude ranging from about 10 MPa to about 30 MPa. The pulse duration can comprise a number of cycles ranging from about 1 cycle to about 20,000 cycles within a timeframe ranging from about 1 microseconds (μs) to about 20 milliseconds (ms). The pulse repetition frequency can comprise a frequency ranging from about 0.1 hertz (Hz) to about 100 Hz. A period of treatment time can range from about 30 seconds to about 300 seconds.

In some embodiments, the hydrogel can include a biocompatible polymer. The biocompatible polymer can comprise substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxy butyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

In some embodiments, the therapeutic species can include at least one of a small molecule, a nucleic acid, a peptide, a protein, a microRNA mimetic, and combinations thereof.

In some embodiments, the linker can include a Diels-Alder reaction product comprising a diene precursor and a dienophile precursor. The diene precursor can include at least one of a substituted or unsubstituted furan, thiophene, or pyrrole. The dienophile precursor can comprise at least one of a substituted or unsubstituted alkene or alkyne.

An exemplary embodiment of the present disclosure provides a method of promoting controlled tissue regeneration in a subject. The method can include disposing, against a tissue of the subject, a material comprising a Diels-Alder reaction product comprising a first precursor and a second precursor, and exposing the material to a first triggering event, thereby initiating a retro-Diels-Alder reaction of the material.

In some embodiments, the method can further comprise encapsulating a therapeutic species within the material. In some embodiments, the method can further comprise coupling a therapeutic species with the material via a Diels-Alder reaction product comprising a third precursor and a fourth precursor different than the first and second precursors.

In some embodiments, the method can further comprise exposing the therapeutic species to a second triggering event, thereby initiating a retro-Diels-Alder reaction of the third and fourth precursor to uncouple the therapeutic species.

In some embodiments, at least one of the first triggering event or the second triggering event can include pulsed waves of acoustic energy.

In some embodiments, the pulsed waves of acoustic energy can include a waveform, a pulse duration, and a pulse repetition frequency.

In some embodiments the waveform can comprise a positive peak pressure amplitude ranging from about 40 megapascals (MPa) to about 100 MPa and a negative peak pressure amplitude ranging from about 10 MPa to about 30 MPa. The pulse duration can comprise a number of cycles ranging from about 1 cycle to about 20,000 cycles within a timeframe ranging from about 1 microseconds (μs) to about 20 milliseconds (ms). The pulse repetition frequency can comprise a frequency ranging from about 0.1 hertz (Hz) to about 100 Hz. A period of treatment time can range from about 30 seconds to about 300 seconds.

In some embodiments, the method can further comprise adjusting the second triggering event to comprise at least one of the waveform, pulse duration, or pulse repetition frequency different from the first triggering event such that the therapeutic species is uncoupled at a different rate than the retro-Diels-Alder reaction of the material.

In some embodiments, the method can further comprise adjusting the second triggering event to comprise at least one of the waveform, pulse duration, or pulse repetition frequency approximately identical to the first triggering event such that the therapeutic species is uncoupled at a similar rate as the retro-Diels-Alder reaction of the material.

In some embodiments, the material can include a biocompatible polymer. The biocompatible polymer can comprise substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxy butyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

In some embodiments, the therapeutic species can include at least one of a small molecule, a nucleic acid, a peptide, a protein, a microRNA mimetic, and combinations thereof.

In some embodiments, the linker can include a Diels-Alder reaction product comprising a diene precursor and a dienophile precursor. The diene precursor can include at least one of a substituted or unsubstituted furan, thiophene, or pyrrole. The dienophile precursor can comprise at least one of a substituted or unsubstituted alkene or alkyne.

These and other aspects of the present disclosure are described in the Detailed Description below and the accompanying drawings. Other aspects and features of embodiments, will become apparent to those of ordinary skill in the art upon reviewing the following description of specific, exemplary embodiments, in concert with the drawings. While features of the present disclosure may be discussed relative to certain embodiments, and figures, all embodiments, of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The accompanying figures, which are incorporated in and constitute a part of this specification, illustrate multiple embodiments of the presently disclosed subject matter and, together with the description, serve to explain the principles of the presently disclosed subject matter: and, furthermore, are not intended in any manner to limit the scope of the presently disclosed subject matter.

FIG. 1 schematically illustrates an example composition positioned within a subject, in accordance with an exemplary embodiment of the present invention.

FIG. 2A schematically illustrates an example composition with a therapeutic species and linker undergoing degradation under external stimulation, in accordance with an exemplary embodiment of the present invention.

FIG. 2B schematically illustrates an example composition with two different therapeutic species joined to two different materials undergoing degradation under external stimulation, in accordance with an exemplary embodiment of the present invention.

FIGS. 3A-3C provide example compositions and precursors for forming linkers and undergoing retro Diels-Alder reaction under external stimulation, in accordance with an exemplary embodiment of the present invention.

FIG. 4A provides FTIR spectra for example chitosan hydrogel and chitosan hydrogel with linkers, in accordance with an exemplary embodiment of the present invention.

FIG. 4B provides DSC curves for example chitosan hydrogel with linkers, in accordance with an exemplary embodiment of the present invention.

FIGS. 4C-4G provide FTIR spectra for example polycaprolactone material and polycaprolactone material with linkers, in accordance with an exemplary embodiment of the present invention.

FIGS. 4H-4K provide DSC curves for example polycaprolactone material and polycaprolactone material with linkers, in accordance with an exemplary embodiment of the present invention.

FIG. 4L provides rheology curves for example chitosan hydrogel with linkers, in accordance with an exemplary embodiment of the present invention.

FIGS. 5A-5F graphically illustrate focused ultrasound dependent release of therapeutic species from example hydrogels and materials, in accordance with an exemplary embodiment of the present invention.

FIG. 6A provides real-time visualization of degradation of example hydrogels under focused ultrasound stimulation, in accordance with an exemplary embodiment of the present invention.

FIG. 6B provides a chart of surrounding temperature change during exposure to various conditions of focused ultrasound, in accordance with an exemplary embodiment of the present invention.

FIG. 6C provides optical profilometry of an example hydrogel with linkers before and after exposure to focused ultrasound, in accordance with an exemplary embodiment of the present invention.

FIGS. 7A and 7B provide cytocompatibility staining of example hydrogels with HeLa cells, in accordance with an exemplary embodiment of the present invention.

FIGS. 8A graphically illustrates metabolic activity of example hydrogels with HeLa cells, in accordance with an exemplary embodiment of the present invention.

FIG. 8B graphically illustrates total cell number of example hydrogels with HeLa cells, in accordance with an exemplary embodiment of the present invention.

FIG. 8C graphically illustrates focused ultrasound dependent release of therapeutic species from example hydrogels and materials immersed for one to four hours in temperatures ranging from 20° C. to 60° C., in accordance with an exemplary embodiment of the present invention.

FIG. 9 provides Gibbs free energy & enthalpy reaction barriers generated for example materials with various linkers and example structures of cycloadducts, in accordance with an exemplary embodiment of the present invention.

FIG. 10 provides an example method of delivering a therapeutic species to a subject, in accordance with an exemplary embodiment of the present invention.

FIG. 11 provides an example method of delivering a therapeutic species to a subject, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature of a “subject” or “patient” may be vasculature of a human or any animal. It should be appreciated that an animal may be a variety of any applicable type, including, but not limited to, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient.

As used herein, the terms “about” or “approximately” for any numerical values or ranges indicate a suitable tolerance. More specifically, “about” or “approximately” can refer to the range of values ±20% of the recited value, e.g. “about 90%” can refer to the range of values from 71% to 99%.

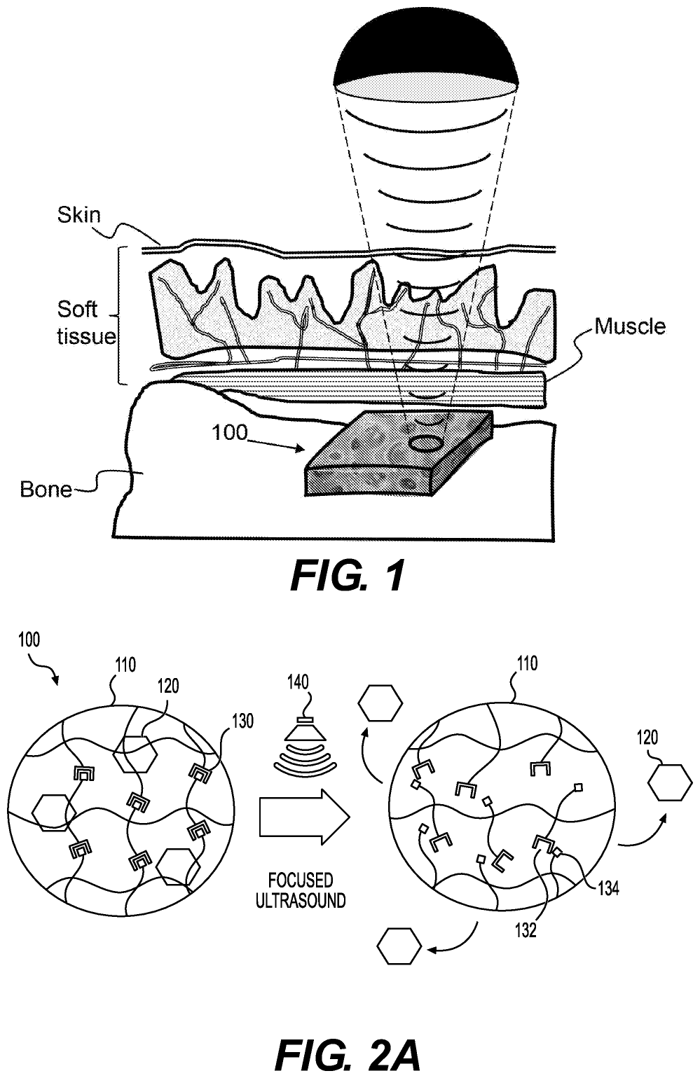

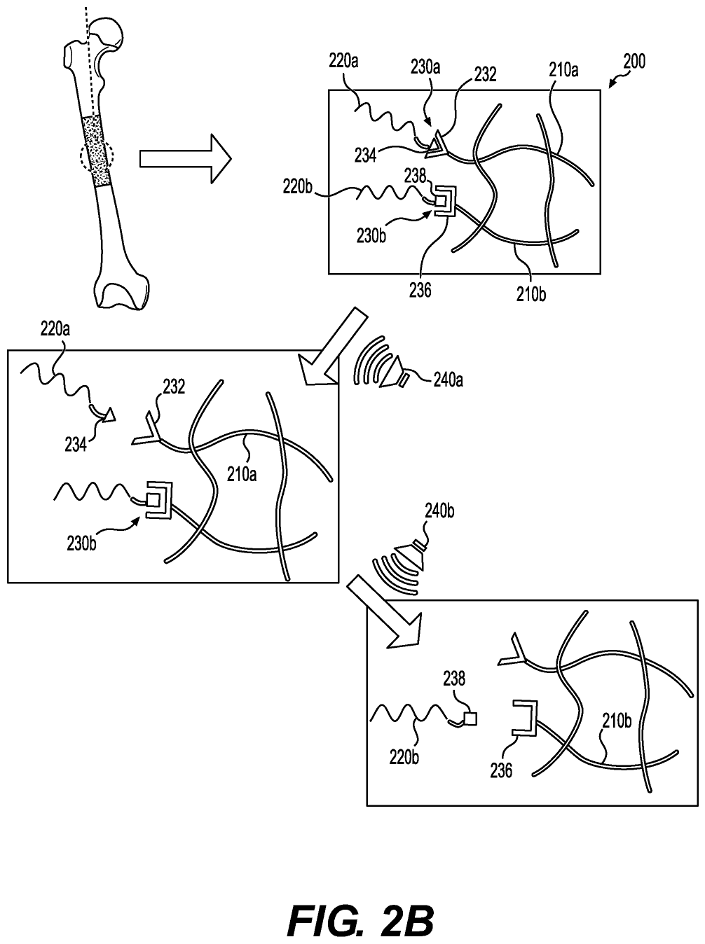

As shown in FIG. 1, an exemplary embodiment of the present invention provides a schematic of composition 100 that can be an implantable structure against a bone of a subject, or can be a drug delivery system, as shown more clearly in FIGS. 2A and 2B.

FIG. 2A provides an example composition 100 including a hydrogel 110 and a therapeutic species 120. Hydrogel 120 can include a linker 130. In some embodiments, linker 130 can join hydrogel 110 together as shown in FIG. 2A. Alternatively, or in addition thereto, linker 130 can join hydrogel 110 directly or indirectly to therapeutic species 120, as illustrated in FIG. 2B and described in more detail below. Linker 130 can include a Diels-Alder cyclo-addition reaction product including a diene 132 and a dienophile 134. As shown in the schematic on the left, hydrogel 110 can be linked via linker 130 and encapsulate therapeutic species 120 when delivered or implanted in subject and before any stimulation by a triggering event 140 sufficient to initiate a retrograde cleavage reaction, or retro Diels-Alder reaction. In some cases, when hydrogel 110 is exposed to triggering event 140, composition 100 is capable of releasing therapeutic species 120 or delivering therapeutic species 120 to a target as illustrated in the right schematic of FIG. 2A.

Turning to FIG. 2B, an example composition 200 includes a first material 210a having a first precursor 232 and a first therapeutic species 120a having a second precursor 234. First precursor 232 and second precursor 234 can together form first linker 230a that is a Diels-Alder cyclo-addition reaction product including a diene and a dienophile. Composition 200 can also include a second material 210b having a third precursor 236 and a second therapeutic species 120b having a fourth precursor 238. Similar to first precursor 232 and second precursor 234, third precursor 236 and fourth precursor 238 can together form a second linker 230b that is a Diels-Alder cyclo-addition reaction product including a diene and a dienophile. As shown in the schematic, first material 210a and second material 210b can together form a polymeric structure, such as a hydrogel or other biocompatible material. Each material 210a, 210b can be linked via functional group crosslinking using well-known crosslinking techniques. Each material 210a, 210b can be directly or indirectly linked with the respective therapeutic species 220a, 220b such that when delivered or implanted in subject and before any stimulation by a first triggering event 240a and/or second triggering event 240b, composition 200 is structurally fixated to a target tissue such as a bone. Upon exposure to first triggering event 240a, first linker 230a can undergo a retrograde cleavage reaction, or retro Diels-Alder reaction, while second linker 230b remains linked, such that only first therapeutic species 220a is released and/or delivered to the target tissue. In the same way, upon exposure to second triggering event 240b, second linker 230a can undergo a retrograde cleavage reaction, or retro Diels-Alder reaction to release and/or deliver second therapeutic species 220b. In some cases, first linker 230a can be substantially different than second linker 230b such that composition 200 releases first therapeutic species 220a at a significantly different energy property (for instance, higher energy or lower energy) of triggering event than the release of second therapeutic species 220b. In certain embodiments, first linker 230a and second linker 230b can be similar such that the first and second triggering events 240a, 240b are close in energy properties (i.e., range of waveform, pulse duration, and/or pulse repetition frequency).

In general, hydrogels are 3D networks of hydrophilic and biocompatible polymers that form from physical crosslinks of individual polymer chains into sponge-like materials that are moldable to any shapes, compressible, and able to be loaded with payloads, as illustrated in FIGS. 2A and 2B. According to some embodiments, hydrogel 110 of composition 100 can be made of biocompatible and biodegradable materials that swell and hold large amounts of water or other fluids when in the uncompressed, 3D network. Alternatively, or in addition thereto, material 210 of composition 200 can be made of a biocompatible structural material that is stiff and can function as a stent or plate.

In some examples, composition 100, 200 can be a polymeric material formed from one or more monomers. In certain embodiments, hydrogel 110 can be formed from precursors 132, 134 having functional groups that form covalent crosslinks that react and gel. In general, precursors are polymerizable and include crosslinker functional groups that react with each other to form polymers made of repeating units. In some cases, hydrogel 110 is made up of precursors 132, 134 that include a Diels-Alder cyclo-addition reaction precursors. In certain examples, material 210 can be made up of a first polymer or material 210a having a first precursor 132 that links with first therapeutic species 120a having a second precursor 134 that together, with the first precursor, forms a Diels-Alder cyclo-addition reaction product.

In various examples, precursors 132, 134, 232, 234, 236, 238 can react by various mechanisms, including chain-growth (addition) or step-growth (condensation) polymerization. In addition, methods for chain-growth and step-growth polymerization of hydrogels can include, without limitation, emulsion polymerization, solution polymerization, suspension polymerization, photopolymerization, ring-opening polymerization, reversible addition-fragmentation chain-transfer polymerization, Diels-Alder cyclo-addition polymerization, plasma polymerization, and precipitation polymerization.

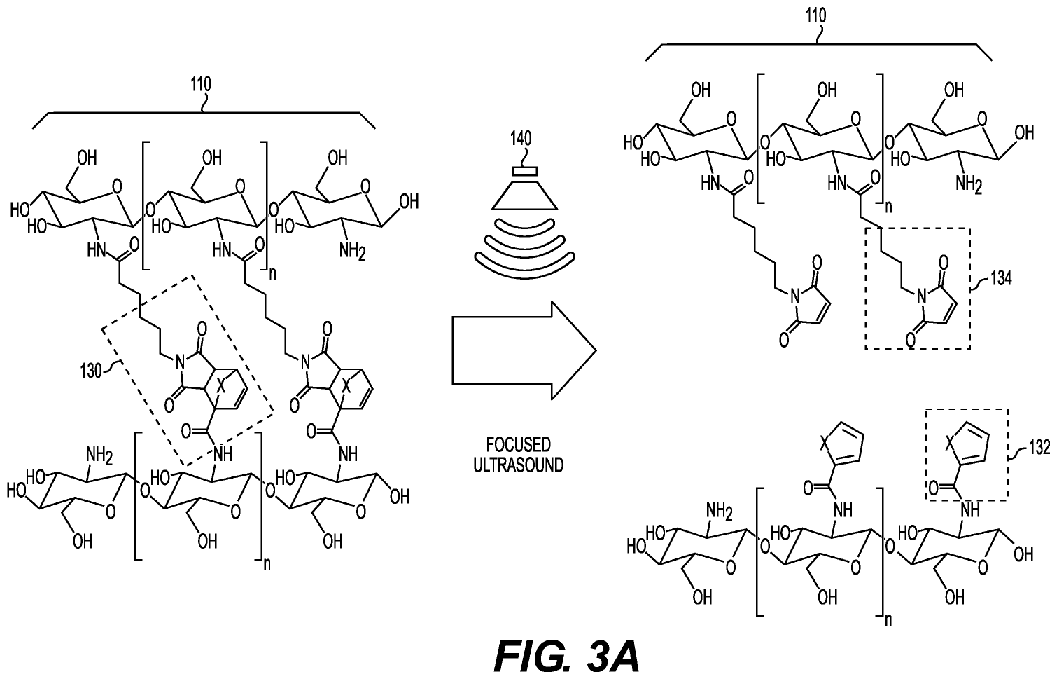

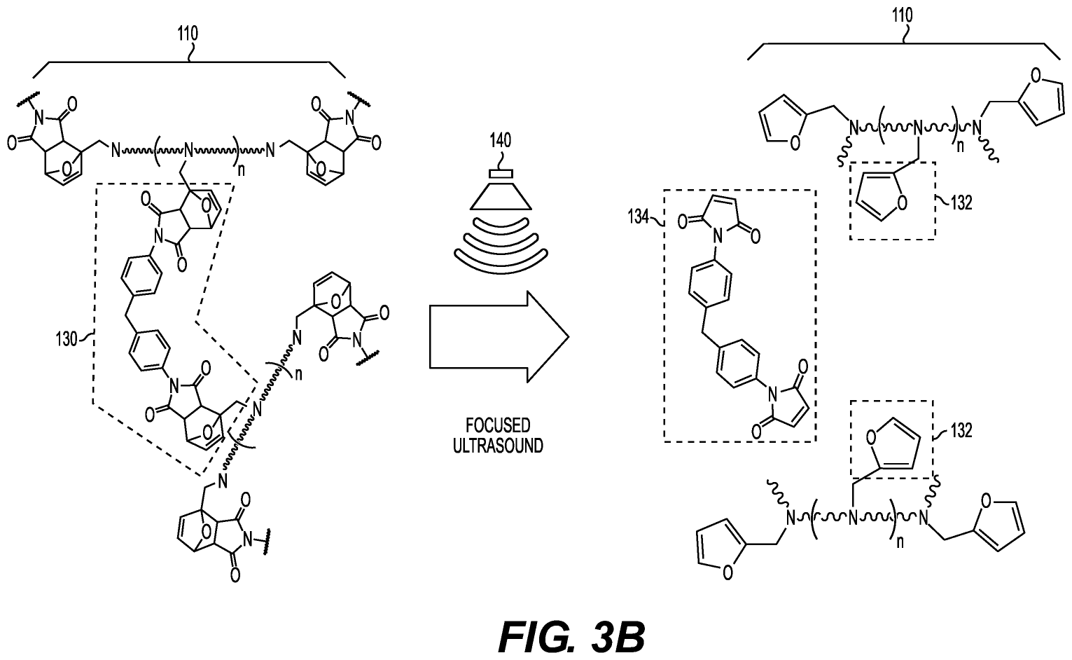

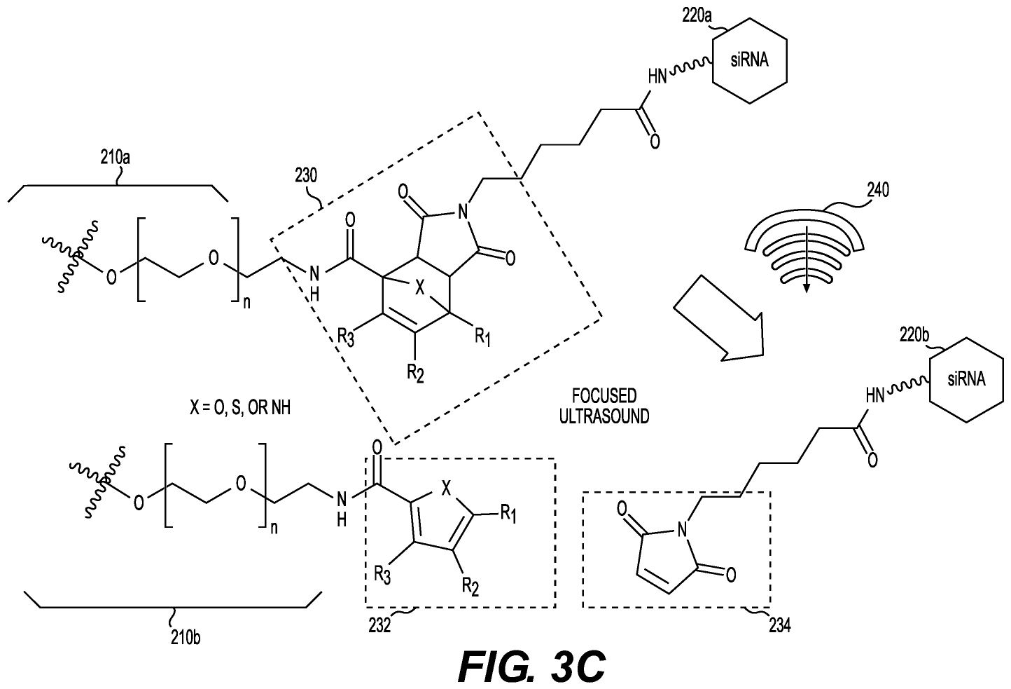

In some embodiments, hydrogel 110 and/or material 210a, 210b can be a biocompatible or biodegradable polymer including, without limitation, polyurethane, polyethylene glycol, poly(methyl vinyl ether-alt maleic acid), polylysine, polyglycolic acid, poly-glycolic acid, poly-L-lactic acid copolymers, polycaprolactive, polyhydroxy butyrate, hydroxyvalerate copolymers, poly-L-lactide, polydioxanone, polycarbonates, and polyanhydrides. Additionally, or alternatively thereto, hydrogel 110 and/or material 210a, 210b can include naturally occurring polysaccharides, such as, for example, chitosan, chitin, cellulose, starch, glycogen, gelatin, amylose, hyaluronic acid, and alginate. Example compositions of hydrogel 110 and/or material 210a, 210b are provided in FIGS. 3A through 3C.

Hydrogel 110 and/or material 210a, 210b size and weight can vary based on the desired viscosity. For instance, a chitosan-based hydrogel having a molecular weight ranging from about 50,000 to about 190,000 Daltons may constitute a low molecular weight hydrogel. In certain embodiments, hydrogel 110 and/or material 210a, 210b can range from about 2 kilodaltons (kDa) to about 400 kDa or more.

In some embodiments, therapeutic species 120, 220a, 220b of composition 100, 200 can be any suitable therapeutic or diagnostic useful for a particular purpose or objective of treating a disease or condition of a patient, including treating a human patient in vivo. For instance, in some examples, the therapeutic species can include a small molecule, a nucleic acid, a peptide, a protein, or combinations thereof.

A small molecule can include a hydrocarbon-based compound, an inorganic compound, or an organometallic compound having a molecular weight between about 100 Daltons to about 1000 Daltons. Small molecule therapeutics can also include saturated compounds having single bonds or unsaturated compounds having double or triple bonds. In some instances, the small molecule can also be linear or cyclic. Nucleic acids can include complex organic substances commonly found in living cells, including, without limitation, DNA or RNA, and their related nucleic acids (e.g., messenger RNA, (“mRNA”), small interfering RNA (“siRNA”), microRNA (“miRNA”), etc.). Nucleic acids can be naturally occurring or synthetic. Similarly, peptides, polypeptides, and proteins can be naturally occurring or synthetic or lab-made. In some embodiments, therapeutic species 120, 220a, 220b can include proteins such as, for example, TNFR2 ECD, humanized IgG, chimeric IgG, modified insulin, human EPO, PEGhuman G-CSF, humanized Fab, human interferon beta-la, factor VIII, factor VIIa, botulinum toxin type A, fluorescein isothiocyanate-tagged albumin (FITC-albumin), suitable microRNAs (e.g., miR-210, miR-148b, miR-21, miR-103/107, miR-92a, miR-16, miR-34a, miR-218, miR-10b, miR-20a, miR-9), miR-181a, miR-29b, miR-10a, and the like) and any suitable monoclonal antibody (e.g., anti-CD3, murine IgG2a, anti-GPIIb/IIIa, chimeric IgGI Fab, anti-CD20, anti-IL2R, anti-RSV, anti-TNF, anti-HER2,anti-CD33, anti-CD52, anti-CD1l1a, anti-VEGF, anti-a4 integrin, anti-IL12/23, anti-IL6R, anti-EPCAM/CD3, anti-RANK-L, anti-BLyS, anti-CTLA-4, and the like) or fusion protein (e.g., aflibercept, rilonacept, alefacept, romiplostim, abatacept/belatacept, denileukin-diftitox, and the like).

According to certain examples, therapeutic species 120, 220a, 220b can be used for the treatment of tissue regeneration. For instance, therapeutic species 120, 220a, 220b can be an osteogenic modulator, a chondrogenic modulator, an endotheliogenic modulator, a myogenic modulator, an anti-cancer agent, an anti-fungal agent, an anti-bacterial agent, or stem cells. Osteogenic modulators can include, without limitation, simvastatin, strontium ranelate, miRNA-26a, miRNA-148b, miRNA-27a, and miRNA-489. Chondrogenic modulators can include, without limitation miRNA-9, miRNA-79, miRNA-140, and miRNA-30A. Some example endotheliogenic modulators can include, without limitation, miRNA-210, miRNA-195, miRNA-155, miRNA-106b, miRNA-93, and miRNA-25. Exemplary embodiments of myogenic modulators can include, without limitation, miRNA-206, miRNA-1, siGDF-8, miRNA-133, miRNA-24, and miRNA-16. Suitable anti-cancer therapeutics, or compositions commonly used in cancer chemotherapy, can include Paclitaxil, Afatinib, Dimaleate, Bortezomib, Carfilzomib, Doxorubicin, Fluorouracil, miRNA-148b, miRNA-135, miRNA-124, miRNA-101, miRNA-29c, miRNA-15a, and miRNA-34. Suitable antifungal agents can include, without limitation, clotrimazole, econazole, miconazole, terbinafine, fluconazole, ketoconazole, and amphotericin. Common antibacterial agents can include penicillin, cephalosporin, tetracycline, aminoglycoside, macrolide, and fluoroquinolone.

In some embodiments, therapeutic species 120, 220a, 220b can also include a theragnostic species, or a species that can be used to diagnose and treat a conditions or disease. Such a theragnostic species can include a diagnostic property such as a fluorescence agent, a phosphorescence agent, radioactivity, MRI activity, or otherwise able to be imaged, tracked, or visualized.

In some embodiments, linker 130 can be any suitable linker that is not inconsistent with the objectives of the present disclosure. As described above, hydrogel 110 and/or material 210 can be made up of two or more precursors that can together undergo a Diels-Alco cycloaddition. Accordingly, any of the precursors 132, 134, 232, 234, 236, 238 may include any suitable Diels-Alder cyclo-addition reaction precursor. As understood by one of ordinary skill in the art, a Diels-Alder reaction is a conjugate addition reaction of a conjugated diene with a dienophile. Certain embodiments of the present disclosure include a suitable diene, such as substituted or unsubstituted alkene. In some embodiments, the suitable diene can include, without limitation, furans, thiophenes, or pyrroles. Without intending to be bound, some example dienes include, without limitation, 1,2-propadiene, isoprene, 1,3-butadiene, 2,4-octanedione, 1,5-cyclooctadiene, norbornadiene, 2-pyrone, dicyclopentadiene, 1H-pyrrole-2-carboxylic acid, 1H-pyrrole-3-carboxylic acid, 3,5-dimethyl-1H-pyrrole-2-carboxylic acid, 1,5-dimethyl-1H-pyrrole-2-carboxylic acid, 2,4,5-trimethyl-1H-pyrrole-3-carboxylic acid, 5-phenyl-1H-pyrrole-2-carboxylic acid, 2,4-dimethyl-1H-pyrrole-3-carboxylic acid, 2,5-dimethyl-1H-pyrrole-3-carboxylic acid, 3-methyl-1H-pyrrole-2-carboxylic acid, 5-(3,4-dimethylphenyl)-2-methyl-1H-pyrrole-3-carboxylic acid, 1-methyl-1H-pyrrole-2-carboxylic acid, 2-methyl-1H-pyrrole-3-carboxylic acid, furan-2- carboxylic acid, furan-3-carboxylic acid, 2-(furan-2-yl)acetic acid, 3-(5-methylfuran-2-yl)propanoic acid, 5-ethylfuran-2-carboxylic acid, 5-isobutyl-2-methylfuran-3-carboxylic acid, 4,5-dimethylfuran-2-carboxylic acid, thiophene-2-carboxylic acid, 4,5-dimethylthiophene-2-carboxylic acid, 3-methylthiophene-2-carboxylic acid, 5-methylthiophene-2-carboxylic acid, 5-phenylthiophene-2-carboxylic acid, 2-(thiophen-2-yl)acetic acid, thiophene-3-carboxylic acid, 2-(thiophen-3-yl)acetic acid, 5-ethylthiophene-2-carboxylic acid, and 5-methyl-4-phenylthiophene-3-carboxylic acid.

In some embodiments, the diene undergoes the Diel-Alder cycloaddition reaction with a suitable dienophile that can include either a substituted or unsubstituted alkene or alkyne. In some embodiments, a suitable dienophile can include, without limitation, acrolein, methyl vinyl ketone, acrylic acid, methyl acrylate, acrylamide, acrylonitrile, methyl acrylate, dimethyl maleate, dimethyl fumarate, maleic anhydride, maleonitrile, butenolide, alpha-methylene gamma-butolactone, N-methylmaleimide, N-ethylmaleimide, dimethyl acetylene dicarboxylate, 6-maleimidohexanoic acid, 2-butenal, 2-Maleimidoacetic acid, 3-Maleimidopropionic acid, 3-Maleimidobenzoic acid, 3-(2,5-Dioxopyrrol-1-yl)hexanoic acid, 4-Maleimidobutyric acid, 4-Maleimidobenzoic acid, 4-(2,5-Dioxopyrrol-1-yl)hexanoic acid, 4-(2,5-dioxo-2,5-dihydro-pyrrol-1-yl)-benzoic acid, 5-Maleimidopentanoic acid, 6-Maleimidohexanoic acid, 6-(3-methyl-2,5-dioxopyrrol-1-yl)hexanoic acid, 6-(2,5-dioxopyrrol-1-yl)-2-methylhexanoic acid, 6-(2,5-Dioxopyrrol-1-yl)-4-methylhexanoic acid, 7-(2,5-Dioxo-2,5-dihydro-1H-pyrrol-1-yl)heptanoic acid, 9-(2,5-dioxopyrrol-1-yl)nonanoic acid, 10)-(2,5-dioxopyrrol-1-yl) decanoic acid, 11-Maleimidoundecanoic acid, 13-(2,5-Dioxo-2,5-dihydro-1H-pyrrol-1-yl)tridecanoic acid, N-(Carboxyheptyl)maleimide, N-(4- Carboxy-3-hydroxyphenyl) maleimide, and α-Maleimidyl-ω-Carboxyl Poly ethylene glycol).

Alternatively, or in addition thereto, the Diels-Alder cyclo-addition reaction product can be a product of a reaction of a dienophile with a substituted or unsubstituted furan, thiophene, or a pyrrole. As would be appreciated by those of skill in the art, hydrogel 110 mechanical strength and reaction time may be adjusted through control of the precursors 132, 134 and functional groups on each precursor 132, 134. The precursors 132, 134 may be mixed and matched to create hydrogel 110 having various features for making composition 100 effective as a drug delivery system. In some embodiments, precursors 132, 134 may react rapidly without the need for catalysts. In some embodiments, precursors 132, 134 may follow click chemistry reactions.

As would be understood by one of skill in the art, first precursor 132 and second precursor 134 can either be a diene or a dienophile, provided that first precursor 132 and second precursor 134 are either present within the hydrogel to function to link hydrogel 110 together or one of the two is within the hydrogel and the other is functionalized with the therapeutic species to link the therapeutic species to the hydrogel via a Diels-Alder cycloaddition, and ultimately to release said therapeutic species via a retro-Diels-Alder reaction. In some embodiments, the therapeutic species can be conjugated to the dienophile, as many suitable dienophiles are cytocompatible: however, the therapeutic species may also be conjugated to the diene. As a non-limiting example, a carbodiimide crosslinker such as 1-Ethyl-3-[3-dimethylaminopropyl] carbodiimide hydrochloride (EDC) or (dicyclohexyl carbodiimide) (DCC) can be used to conjugate carboxylic acids (—COOH) on the dienophile to amines (—NH₂) on the therapeutic species via amide bond formation.

In the same way, with two or more materials 210a, 210b and/or two or more therapeutic species 220a, 220b, two or more combinations of diene and dienophiles can be used to generate two or more unique Diels-Alder cycloaddition reactions that undergo retro-Diels-Alder reactions under differing triggering events. As such, first precursor 232, second precursor 234, third precursor 236, and fourth precursor 238 may independently be a suitable diene and/or a dienophile described above such that two differing resulting cyclohexene or heterocyclic Diels-Alder reaction products are formed.

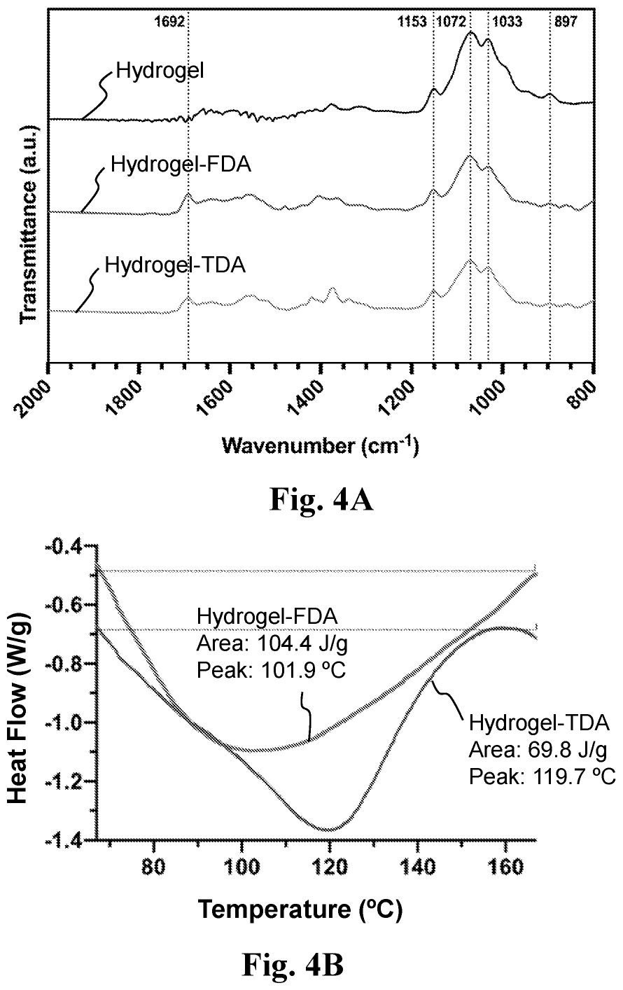

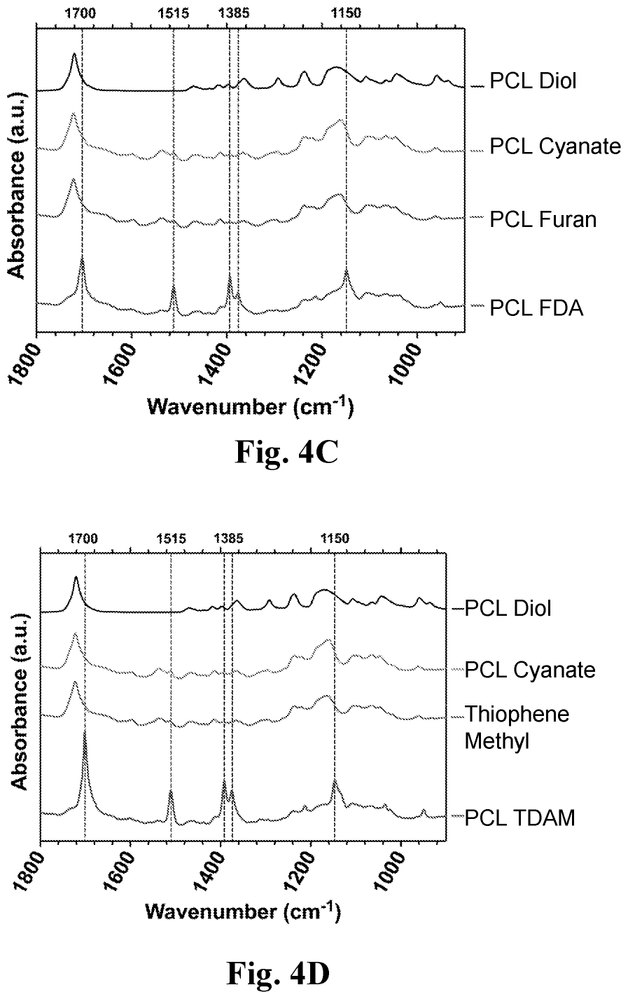

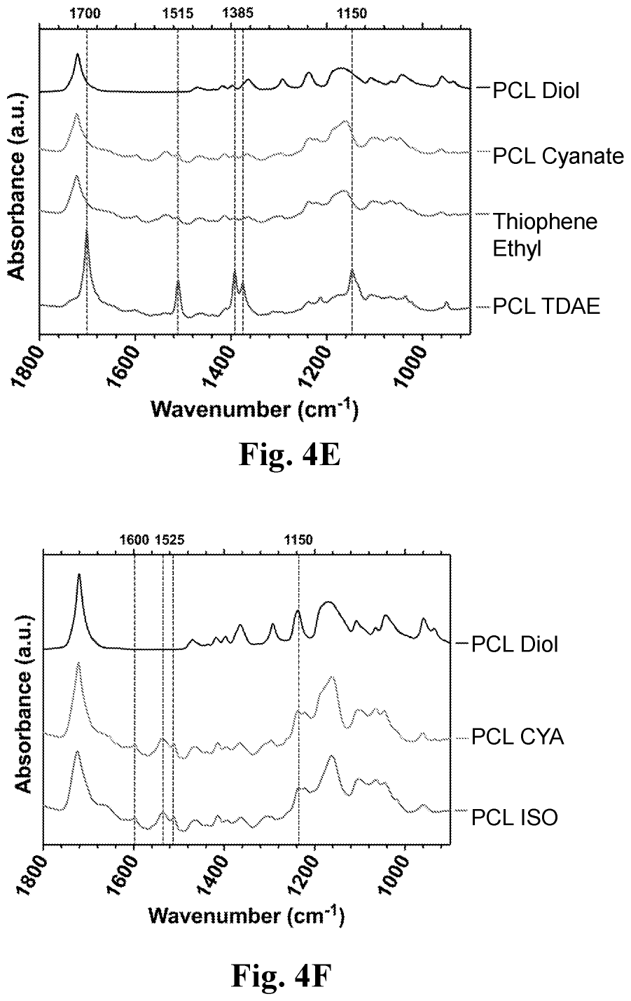

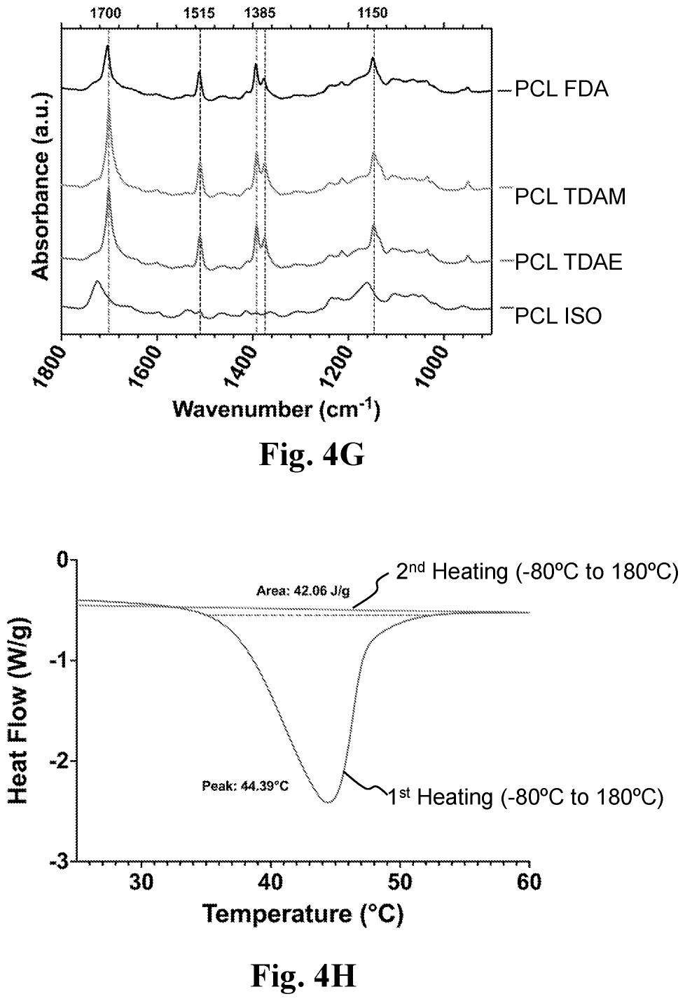

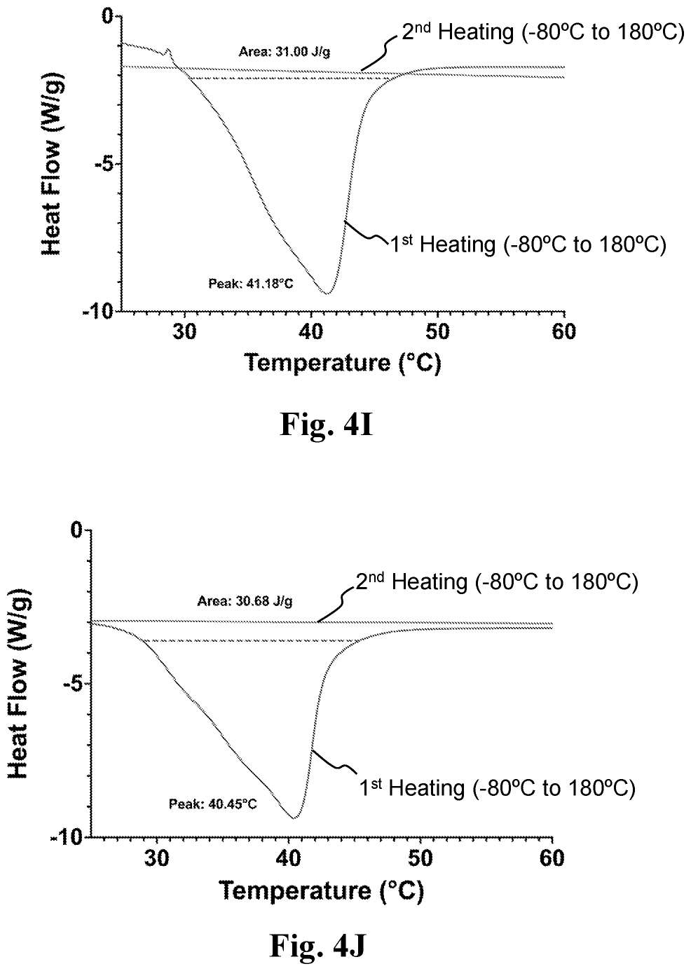

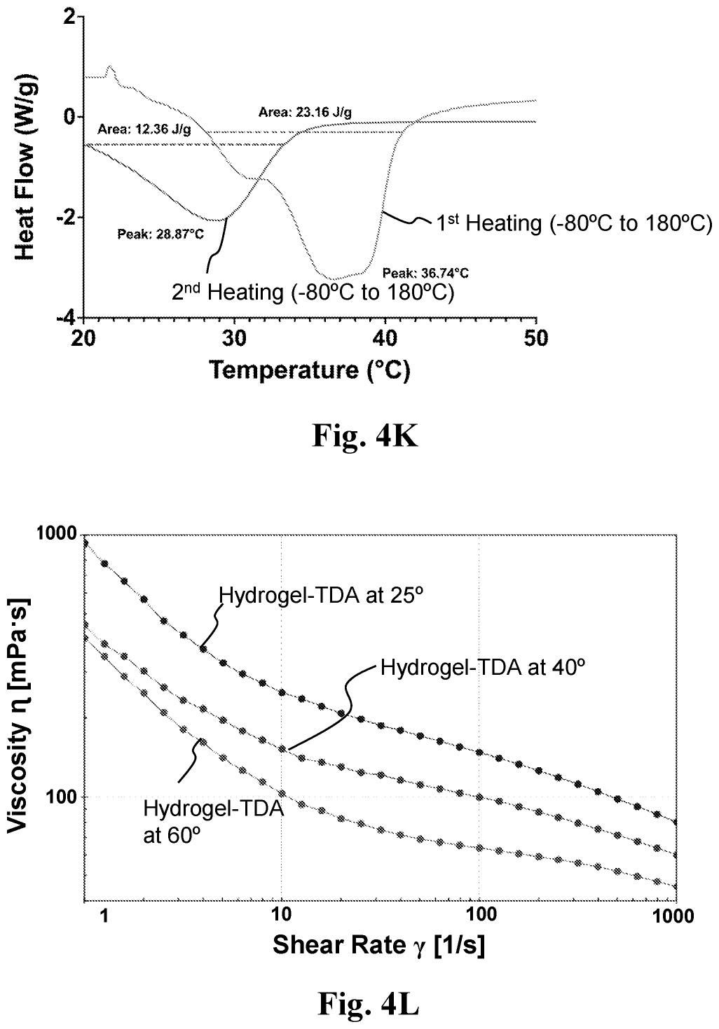

The FTIR spectra of crosslinking hydrogel 110 with linker 130 is shown in FIG. 4A. According to some examples, the extent of crosslinking of hydrogel 110 with linker 130, where the extent of crosslinking with precursors 132, 134 resulting in linker 130 results in a diminished intensity of the FTIR peak for hydrogel 110. As an example, broad peaks around 1153 cm⁻¹, 1072 cm⁻¹, 1033 cm⁻¹, and 897 cm⁻¹ can be attributed to hydrogel 110. As shown in FIG. 4A, crosslinking hydrogel 110 with linker 130 can result in a peak around 1692 cm⁻¹.

Additionally, differential scanning calorimetry (DSC) curves of hydrogel 110 crosslinked with linker 130 are provided in FIG. 4B. According to some examples, hydrogel 110 crosslinked with linker 130 may experience a transition in crosslinking with heat flow. In some embodiments, the crosslinking of hydrogel 110 and linker 130 may experience a change at a temperature of about 101.9° C., as provided as an example in FIG. 4B. As an alternative, or in addition thereto, hydrogel 110 and linker 130 having a different Diels-Alder cyclo-addition reaction product or variation in extent of crosslinking, the material may experience a change at a temperature of about 119.7° C. As would be appreciated by one of skill in the art, adjusting the Diels-Alder cyclo-additional reaction product or the degree of crosslinking may adjust the temperature and heat flow associated with the transition in materials that leads to releasing therapeutic species 120.

FIGS. 4C through 4G provide FTIR spectra of crosslinking material 210 with linker 130. According to some examples, material 210 can be adjusted to vary the extent of crosslinking with the precursors 232, 234, 236, and 238. FTIR spectra of FIGS. 4C through 4G demonstrate an example material 210 as a diol (top spectra in each), example material 210 as a cyanate, and crosslinked samples of material 210 with first linker 130a and/or second linker 130b. Between the diol and the two crosslinked materials, stretches for aromatic rings are present at 1650-1580 cm⁻¹. With various linkers, 130a, 130b, small bumps at 3350 cm⁻¹ are due to amide N-H stretches. Aldehyde versus aliphatic ketone C═O stretches can be found at 1700 cm⁻¹. In some examples, an increase of C═C and C═O bonds is present at 1515 cm⁻¹. whereas 1395-1375 cm⁻¹ is C—H bending from cycloaddition. Small differences from 1250-1020 cm⁻¹ are due to C—N bonds. Stronger Diels-Alder peak at 1150 cm⁻¹ is due to C—O stretching.

In some embodiments, the Diels-Alder cyclo-addition product can further undergo a retro, or reverse Diels-Alder reaction, where linker 130 can fragment and initiate release of therapeutic species 120. As illustrated in FIG. 2A, hydrogel 110 can release therapeutic species 120 in a controlled manner when linker 130 undergoes the retro Diels-Alder reaction. In some examples, retro Diels-Alder fragmentation may result in the same pattern of bond breaking as bond formation that is capable of restructuring and reforming linker 130. As shown in FIG. 3A, biocompatible 6-maleimidohexanoic acid (a dienophile) can undergo a general cyclo-addition reaction with a furan (where X═O), a thiophene (where X═S), or a pyrrole (where X═NH), and where n can be any integer from about 10 to about 2200 while maintaining precursor functional groups after stimulation. In certain examples, the retro Diels-Alder fragmentation may result in different functional groups within hydrogel 110 than first precursor 132 and second precursor 134 as a result of stimulation. Additionally, or alternatively thereto, the fragmentation may result in a hetero Diels-Alder fragmentation, where the bond breaking incorporates forming a heterocycle including oxygen, nitrogen, or sulfur. In some examples, the retro Diels-Alder can be irreversible where by-products such as, for example, CO or CO₂, can be generated and released.

Returning back to FIG. 2A, according to some embodiments, hydrogel 110, and more specifically, linker 130 can undergo a retro Diels-Alder reaction under focused ultrasound external stimulus to release therapeutic species 120. In general, focused ultrasound is a non-invasive technique that uses non-ionizing ultrasonic waves to heat, ablate, and/or cavitate tissue at a controlled adjustable depth. In general, the focused ultrasound can interact with the hydrogel 110 and form cavities or bubbles within hydrogel 110. Differing from ultrasonic imaging, focused ultrasound uses pulsed waves to achieve necessary thermal or mechanical doses to the target. Additionally, focused ultrasound permits ultrasonic waves to propagate through many layers of tissue (e.g., skin, soft tissue, muscle, etc.,) while only focusing on the target, as illustrated in FIG. 1.

In some embodiments, pulsed waves of acoustic energy from the focused ultrasound can include adjustable parameters such as pulse repetition frequency, pulse duration, peak pressure amplitude, and duty cycle. In addition, the parameters of each can be adjusted for desired results for releasing the therapeutic species 120 from the hydrogel 110 and/or sequentially releasing therapeutic species 220a, 220b from first and second material 210a, 210b, respectively.

In some embodiments, focused ultrasound stimulation can have a range of pulse repetition frequencies in order to initiate retrograde cleavage reaction of the linker 130, 230. For instance, pulse repetition frequencies may vary from about 0.1 hertz (Hz) to about 100 Hz (e.g., from about 0.1 Hz to about 0.5 Hz, from about 0.5 Hz to about 1 Hz, from about 1 Hz to about 5 Hz, from about 5 Hz to about 10 Hz, from about 10 Hz to about 15 Hz, from about 15 Hz to about 20 Hz, from about 20 Hz to about 30 Hz, from about 30) Hz to about 40) Hz, from about 40) Hz to about 50 Hz, from about 50 Hz to about 60 Hz, from about 60 Hz to about 70) Hz from about 70 Hz to about 80 Hz, from about 80 Hz to about 90 Hz, from about 90 Hz to about 100 Hz, and any range in between, e.g., from about 23 Hz to about 89.6 Hz).

Depending on target tissue depth, size, and injury level, the focused ultrasound frequency can increase or decrease to control the extent of retrograde cleavage reaction of the linker 130, 230. For instance, applying the methods described herein to a composition 100, 200 positioned 10 mm beneath the skin may require parameters of the fUS transducer compared to a composition 100, 200 positioned 50 mm or more beneath the skin. In addition, focused ultrasound stimulation may have a pulse duration ranging from about 1 us to about 1000 ms (e.g., from about 1 μs to about 100 μs, from about 100 μs to about 200 μs, from about 200 μs to about 300 μs, from about 300 μs to about 400 μs, from about 400 μs to about 500 μs, from about 500 μs to about 600 μs, from about 600 μs to about 700 μs, from about 700 μs to about 800 μs, from about 800 μs to about 900 μs, from about 900 μs to about 1 ms, from about 1 ms to about 100 ms, from about 100 ms to about 200 ms, from about 200 ms to about 300 ms, from about 300 ms to about 400 ms, from about 400 ms to about 500 ms, from about 500 ms to about 600 ms, from about 600 ms to about 700 ms, from about 700 ms to about 800 ms, from about 800 ms to about 900 ms, from about 900 ms to about 1 s).

In some embodiments, focused ultrasound stimulation can have a pulse duration ranging from about 1 cycle to about 1,000,000 cycles for a specific duration of time and at a specific frequency (e.g., from about 1 to about 10 cycles, from about 10 cycles to about 100 cycles, from about 100 cycles to about 500 cycles, from about 500 cycles to about 1,000cycles, from about 1,000 cycles to about 2,000 cycles, from about 2,000 cycles to about 4,000 cycles, from about 4,000 cycles to about 8,000 cycles, from about 8,000 cycles to about 16,000 cycles, from about 16,000 cycles to about 20,000 cycles, from about 20,000 cycles to about 30,000 cycles, from about 30,000 cycles to about 40,000 cycles, from about 40,000 cycles to about 50,000 cycles, from about 50,000 cycles to about 60,000 cycles, from about 60,000 cycles to about 70,000 cycles, from about 70,000 cycles to about 80,000 cycles, from about 80,000 cycles to about 90,000 cycles, from about 90,000 cycles to about 100,000 cycles, from about 100,000 cycles to about 200,000 cycles, from about 200,000 cycles to about 300,000 cycles, from about 300,000 cycles to about 400,000 cycles, from about 400,000 cycles to about 500,000 cycles, from about 500,000 cycles to about 600,000 cycles, from about 600,000 cycles to about 700,000 cycles, from about 700,000 cycles to about 800,000 cycles, from about 800,000 cycles to about 900,000 cycles, from about 900,000 cycles to about 1,000,000 cycles, and any range in between, e.g., from about 1,568 cycles to about 160,504 cycles). For instance, in some embodiments, a pulse of 20 ms repeated at 1 Hz for 5 minutes may sufficiently induce retrograde cleavage reaction of linker 130 to effectively release therapeutic species 120.

Additionally, focused ultrasound stimulation may have an amplitude corresponding to peak acoustic pressures ranging from about 100 mPa to about 1 Pa. A peak pressure of about 100 mV can correlate to about 8 MPa peak positive and about 6 MPa peak negative. Similarly, a peak pressure of about 300 mV can correlate to a peak positive pressure of about 37 MPa and a peak negative pressure of about 16 MPa. A peak pressure of about 500 mV can correlate to a peak positive pressure of about 69 MPa and a peak negative pressure of about 21 MPa. A peak pressure of about 700 mV can correlate to a peak positive pressure of about 106 MPa and a peak negative pressure of about 28 MPa. In some embodiments, focused ultrasound may have a pressure amplitude or intensity with a maximum value of about 40 MPa to about 100 MPa at positive peak pressure and a magnitude of about 10 MPa to about 30 MPa at negative peak pressure.

In some embodiments, focused ultrasound stimulation can have a range of treatment time ranging from about 30 seconds to about 300 seconds (e.g., from about 30 s to about 40 s, from about 40 s to about 50 s, from about 50 s to about 60 s, from about 60 s to about 70 s, from about 70 s to about 80 s, from about 80 s to about 90 s, from about 90 s to about 100 s, from about 100 s to about 110 s, from about 110 s to about 120 s, from about 120s to about 130 s, from about 130 s to about 140 s, from about 140 s to about 150 s, from about 150 s to about 160 s, from about 160 s to about 170 s, from about 170 s to about 180 s, from about 180 s to about 190 s, from about 190 s to about 200 s, from about 200 s to about 210 s, from about 210 s to about 220 s, from about 220s to about 230 s, from about 230 s to about 240 s, from about 240 s to about 250 s, from about 250 s to about 260 s, from about 260 s to about 270 s, from about 270 s to about 280 s, from about 280 s to about 290 s, from about 290 s to about 300 s, and any range in between, e.g., from about 34 s to about 206 s).

Depending on linker 130 and the respective precursors 132, 134 used in hydrogel 110, or first linker 130a and second linker 130b used in materials 210a, 210b, the frequency of stimulation can increase or decrease to control the extent of fragmentation. In addition, focused ultrasound stimulation may have a pulse duration ranging from about 1 μs to about 1 s (e.g., from about 1 μs to about 100 μs, from about 100 μs to about 200 μs, from about 200 us to about 300 μs, from about 300 μs to about 400 μs, from about 400 μs to about 500 μs, from about 500 μs to about 600 μs, from about 600 μs to about 700 μs, from about 700 μs to about 800 μs, from about 800 μs to about 900 μs, from about 900 μs to about 1 ms, from about 1 ms to about 100 ms, from about 100 ms to about 200 ms, from about 200 ms to about 300 ms, from about 300 ms to about 400 ms, from about 400 ms to about 500 ms, from about 500 ms to about 600 ms, from about 600 ms to about 700 ms, from about 700 ms to about 800 ms, from about 800 ms to about 900 ms, from about 900 ms to about 1 s).

In some embodiments, focused ultrasound stimulation can have a pulse repetition frequency ranging from about 0.1 Hz to about 1000 Hz for a specific duration of time and at a specific frequency (e.g., from about 0.1 Hz to about 1 Hz, from about 1 Hz to about 100 Hz, from about 100 Hz to about 200 Hz, from about 200 Hz to about 300 Hz, from about 300 Hz to about 400 Hz, from about 400 Hz to about 500 Hz, from about 500 Hz to about 600 Hz, from about 600 Hz to about 700 Hz, from about 700 Hz to about 800 Hz, from about 800 Hz to about 900 Hz, from about 900 Hz to about 1000 Hz). For instance, in some embodiments, a pulse of 20 ms repeated at 1 Hz for 5 minutes may sufficiently fragment hydrogel 110 to effectively release therapeutic species 120.

Additionally, focused ultrasound may have an amplitude corresponding to peak voltage ranging from about 100 mV to about 1 V. A peak pressure of about 100 mV can correlate to about 8 MPa peak positive and about 6 MPa peak negative. Similarly, a peak pressure of about 300 mV can correlate to a peak positive pressure of about 37 MPa and a peak negative pressure of about 16 MPa. A peak pressure of about 500 mV can correlate to a peak positive pressure of about 69 MPa and a peak negative pressure of about 21 MPa. A peak pressure of about 700 mV can correlate to a peak positive pressure of about 106 MPa and a peak negative pressure of about 28 MPa. In some embodiments, focused ultrasound may have a pressure amplitude or intensity with a maximum value of about 10 MPa to about 30 MPa at positive pressure and a magnitude of about 15 MPa to about 25 MPa at negative pressure.

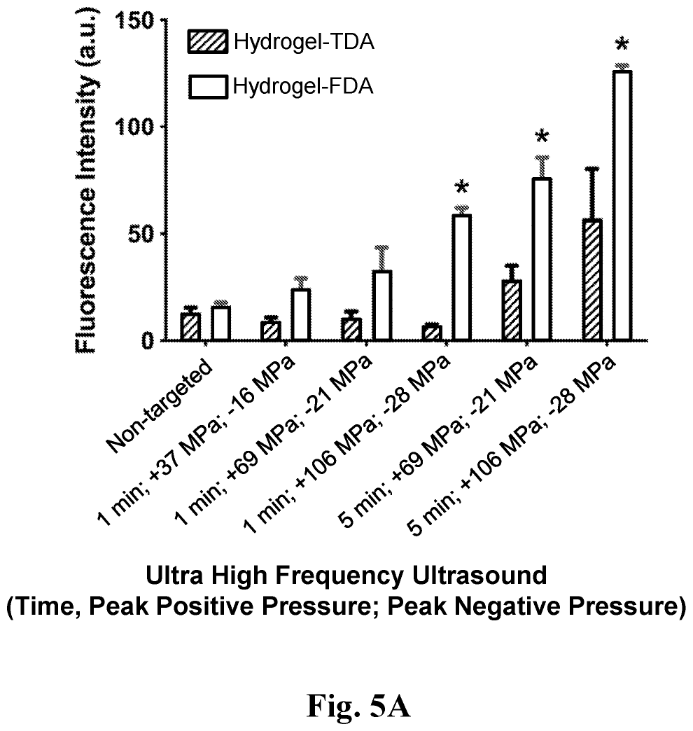

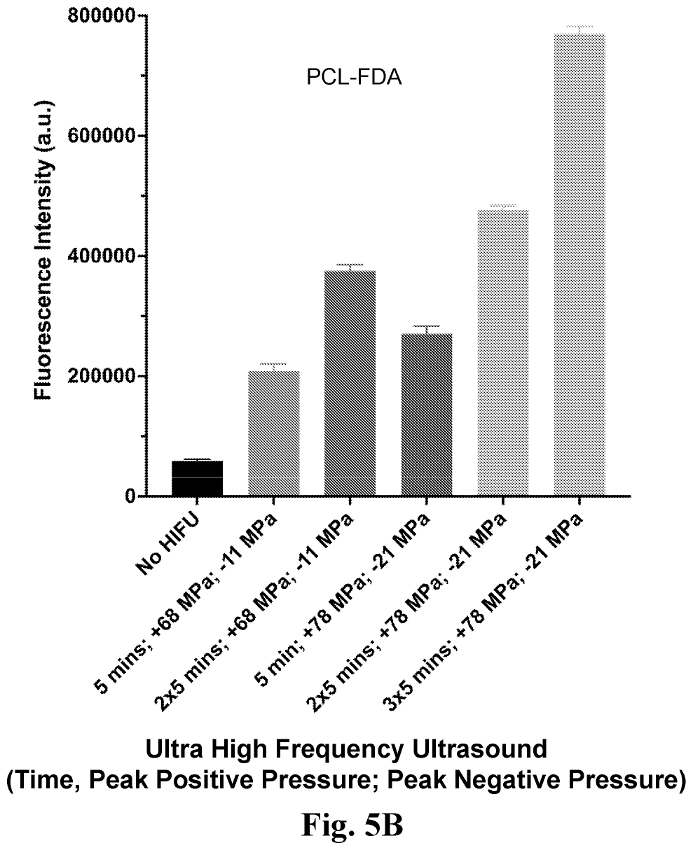

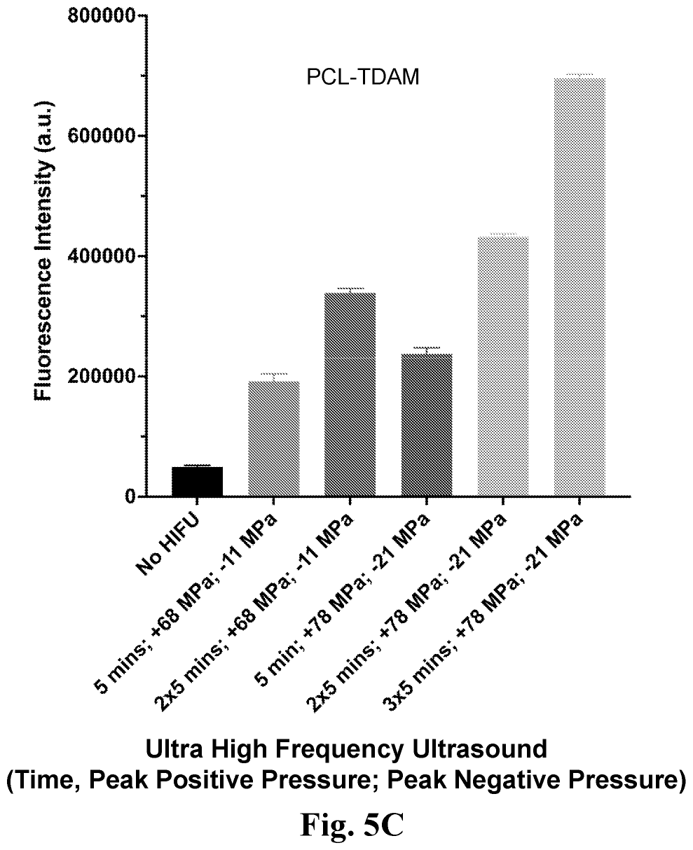

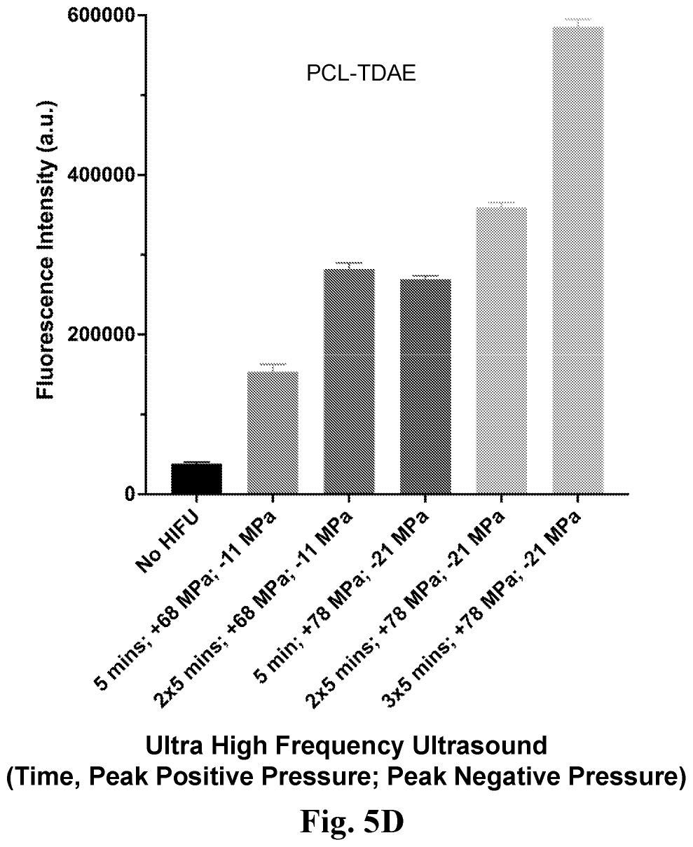

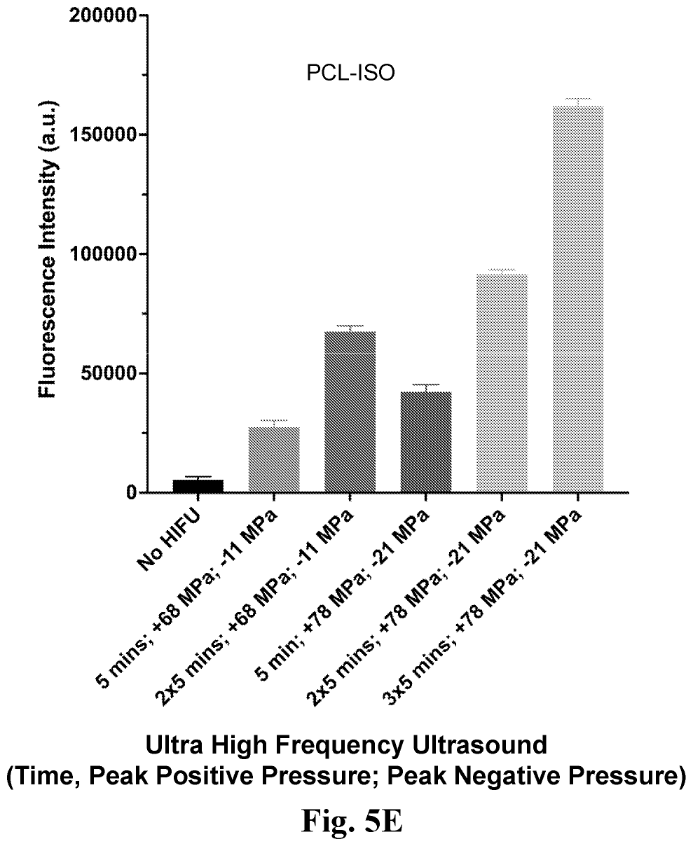

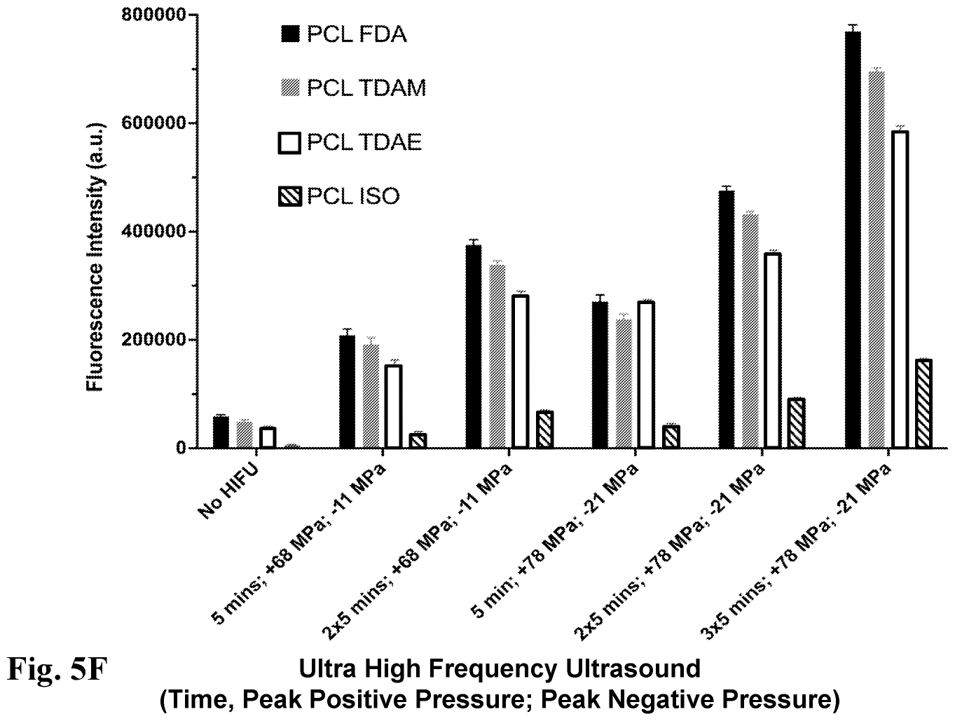

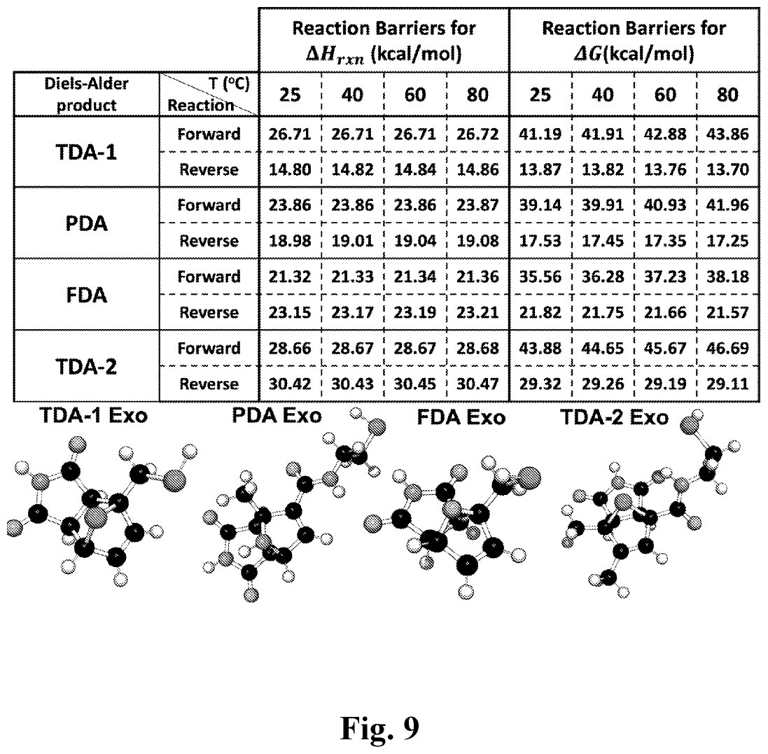

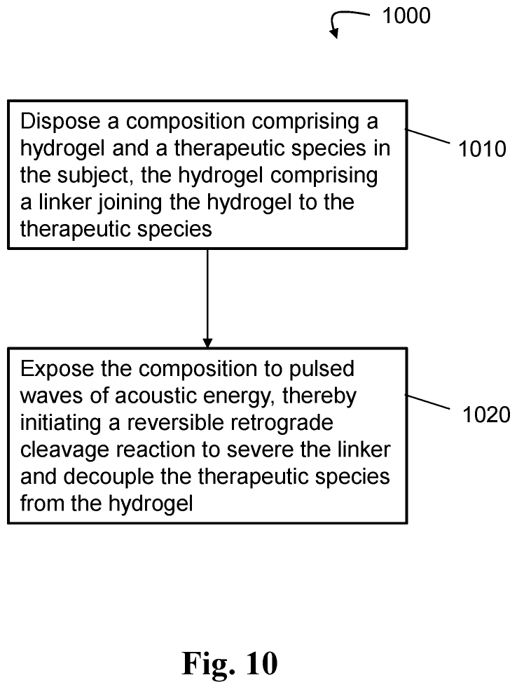

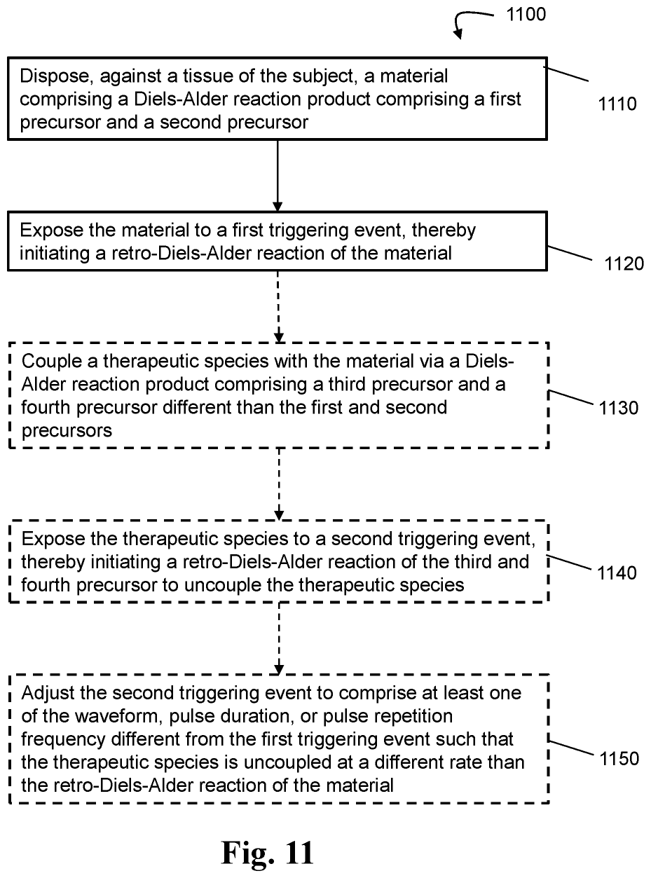

As would be appreciated by those of skill in the art, the fragmentation of various linkers 130, 230a, 230b can vary as the energy barriers for breaking such bonds within linker 130, 230a, 230b depends on the precursors crosslinked. FIG. 9 provides example Gibbs free energy & enthalpy reaction barriers generated for example materials with various linkers. In some embodiments, retro Diels-Alder cycloadduct with a furan-based linker may have a higher release rate or greater fractionality rate than a thiophene-based linker. Accordingly, in some embodiments, the rate of release of therapeutic species 120, 220a, 220b may be greater for linker 130, 230a, 230b having a precursor having a furan compared to a thiophene. As shown in FIGS. 5A and 5F, the focused ultrasound release of a therapeutic species was shown to be higher, as measured by fluorescence intensity, for various linkers having a precursor comprising a furan.

In some embodiments, focused ultrasound can pass through tissue when focused on stimulating composition 100, 200 within a subject. In general, attenuation, or reduction of amplitude, of ultrasound waves in soft tissue depends on the initial frequency of the ultrasound and the distance it has to travel. For instance, in soft tissue, the greater the frequency of the ultrasound waves, the higher the attenuation. In some embodiments, focused ultrasound can image deeper with a lower frequency transducer. In some embodiments, when the ultrasound travels further into the tissue, the ultrasound is attenuation is higher.

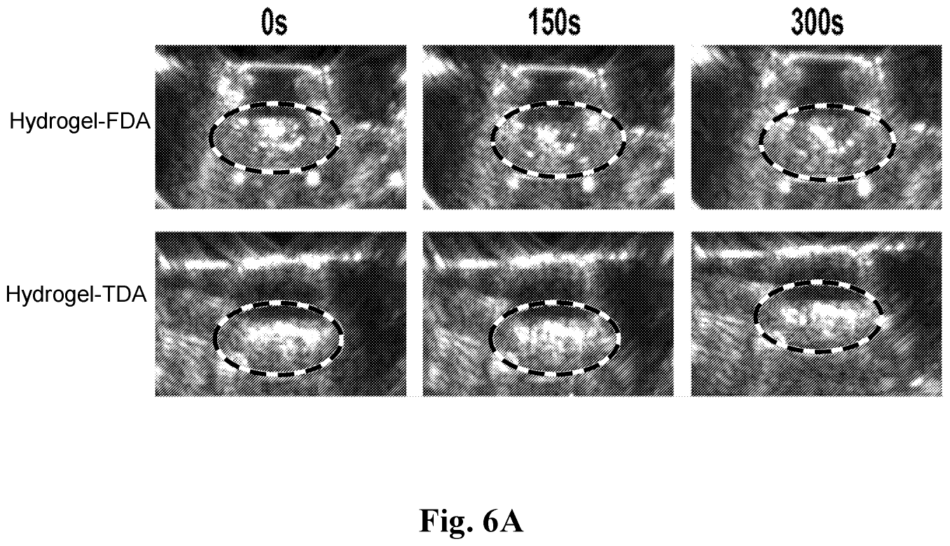

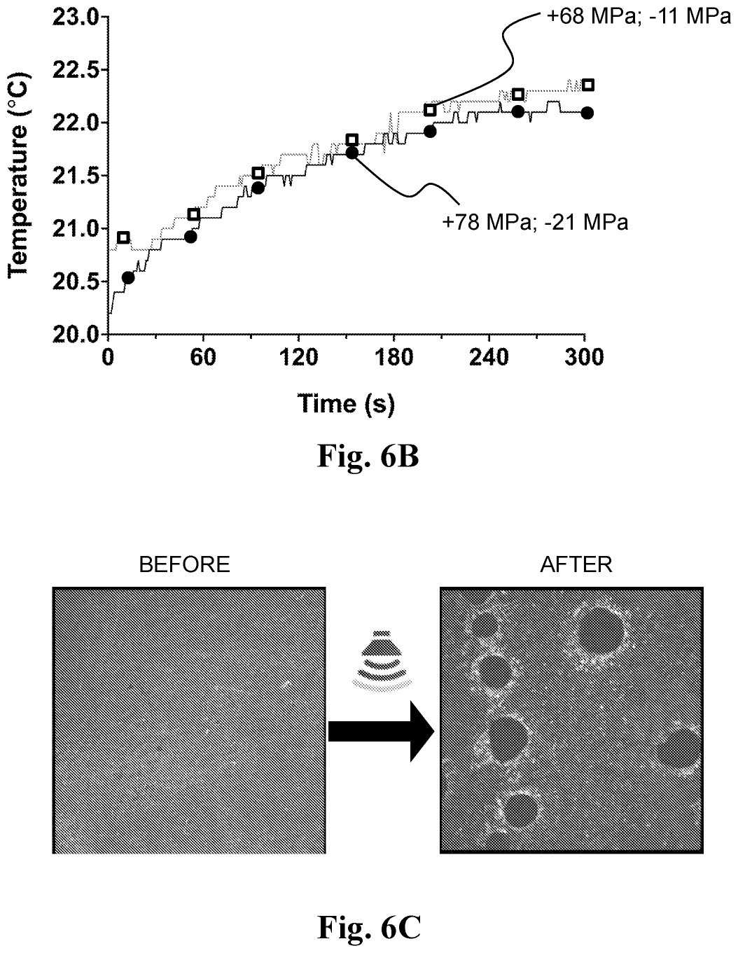

As shown in FIG. 6A, ultrasound-mediated release of a therapeutic species from the composition may be visualized in real-time using ultrasound imaging in combination with the focused ultrasound. Additionally, release of a therapeutic species may further be visualized when a component within composition 100, 200, or the therapeutic species 120, 220a, 220b itself if also luminescent (e.g., fluorescent or phosphorescent), radioactive, MRI active, or otherwise capable of being imaged or tracked. Importantly, using focused ultrasound can limit off-target effects to surrounding tissue. As shown in FIG. 6B, after 5 minutes of focused ultrasound at various parameters prevented an increase in temperature to surrounding tissue more than 2 degrees Celsius. When delivering a high-intensity focused ultrasound of 700 mV with peak amplitudes of +78 MPa and −21 MPa, the initial temperature of the surrounding tissue measured at 20.2° C., and after 5 minutes (300 seconds), the end temperature of the surrounding tissue measured at 22.1° C. (an increase of 1.9° C.). Similarly, when delivering a high-intensity focused ultrasound of 300 mV with peak amplitudes of +68 MPa and −11 MPa, the initial temperature of the surrounding tissue measured at 20.8° C., and after 5 minutes (300 seconds), the end temperature of the surrounding tissue measured at 22.3° C. (an increase of 1.5° C.).

FIG. 6C provides optical profilometry of composition 100, 200 before and after exposure to 700 mV focused ultrasound with peak amplitudes of +78 MPa and −21 MPa. As shown, before exposure, composition 100, 200 has a smooth and continuous surface. After exposure to fUS for 10 minutes, composition 100, 200 begins to present gaps in the surface where therapeutic species 120, 220a, 220b may be released to the target.

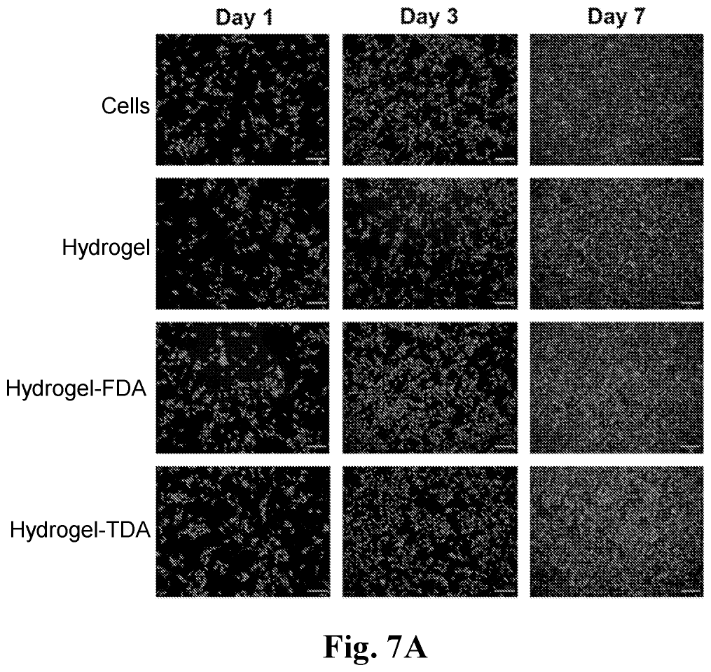

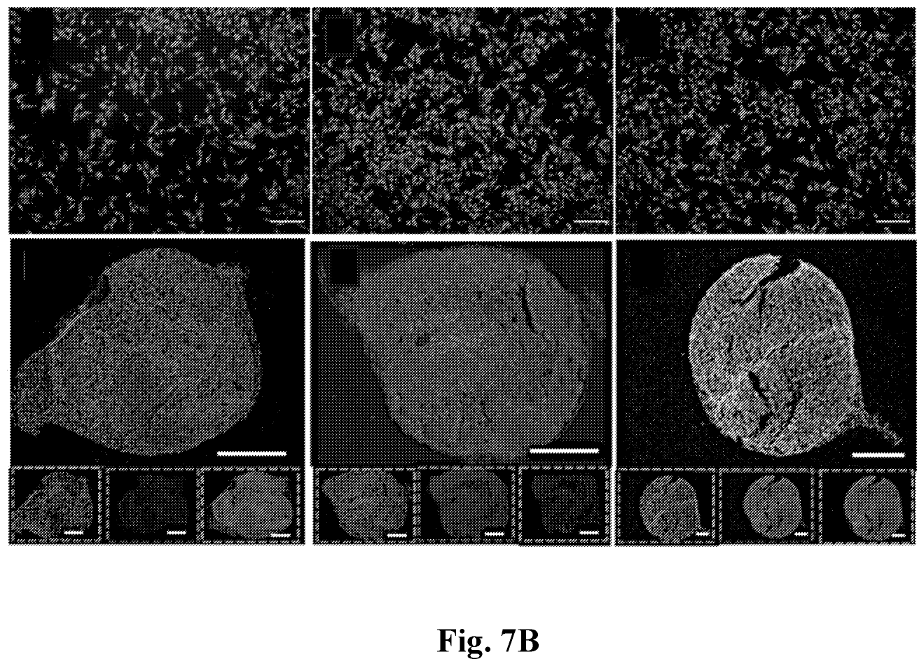

FIG. 7A provides images for cell viability and proliferation assays after exposing cells to composition 100, 200 after 1 day, 3 days, and 7 days. Similarly, FIG. 7B shows images of transfection of at least one therapeutic species 120, 220a, 220b delivered via composition 100, 200.

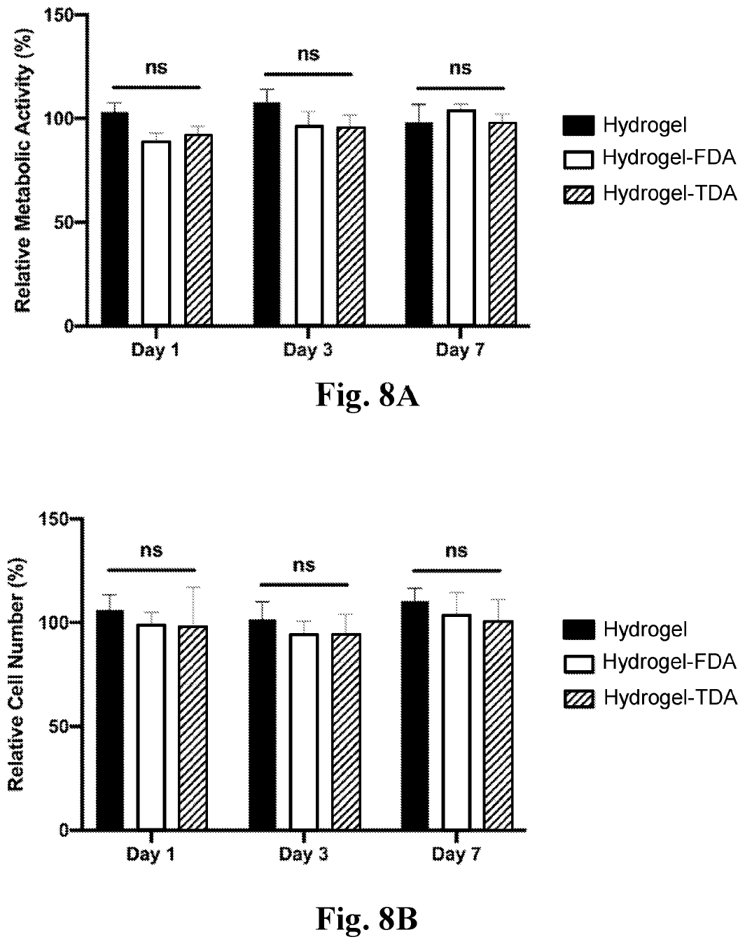

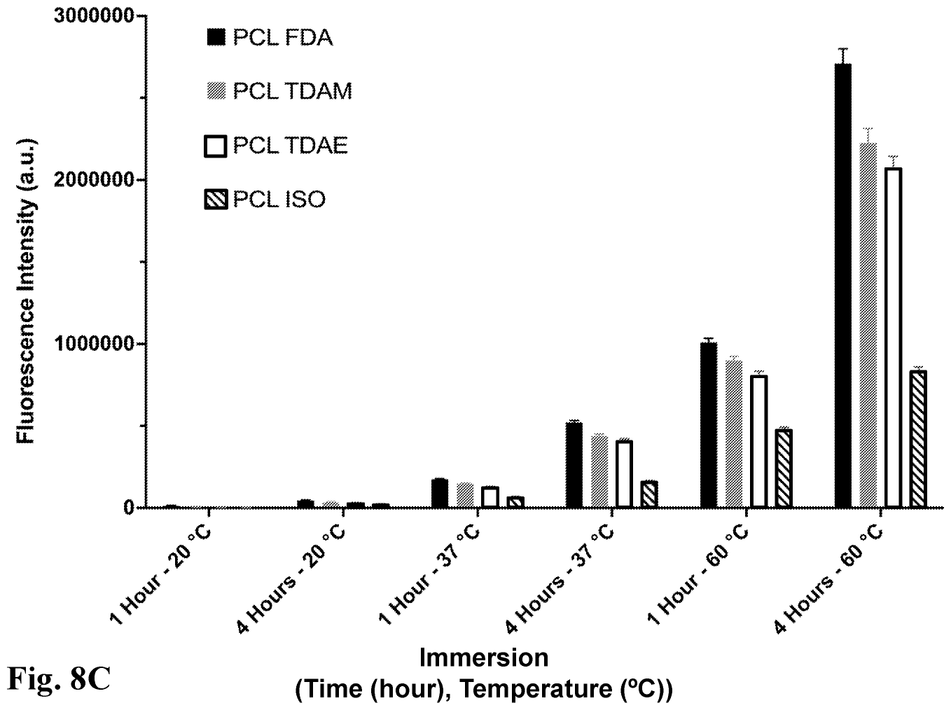

Cytocompatibility of compositions 100, 200 are provided in FIGS. 8A and 8B. As shown, after 7 days, hydrogel or material 110, 210 alone (without linker), and with linker (FDA or TDA) show near 100% relative metabolic activity and 100% relative cell number or viability. Compositions 100, 200 did not appear to induce any cytotoxicity.

FIG. 8C graphically illustrates focused ultrasound dependent release of therapeutic species from example hydrogels and materials immersed for one to four hours in temperatures ranging from 20° C. to 60° C. FIG. 9 provides Gibbs free energy & enthalpy reaction barriers generated for example materials with various linkers and example structures of cycloadducts. As shown, example Diels-Alder linkers such as 4,5-Dimethyl-N-(2-sulfanylethyl)-2-thiophenecarboxamide (TDA-1), pyrrole-2-carboxylic acid (PDA), 2-furanmethanethiol (FDA), 2-Thiophenemethanethiol (TDA-2) diene can have a wide range of reaction barriers for the forward reaction (Diels-Alder reaction product) and the reverse reaction (retro-Diels-Alder reaction) based DA cycloadduct linkers.

In some embodiments, composition 100, 200 may release therapeutic species 120, 220a, 220b under stimulation specific to an energy barrier of breaking linker 130, 230a, 230b. Additionally, composition 100, 200 can undergo a retro Diels-Alder fragmentation to release therapeutic species 120, 220a, 220b under focused ultrasonic stimulation in combination with various other forms of stimulation, including, without limitation, electrical stimulation, heat, light, magnetic field, or chemical stimulation. For instance, combining focused ultrasound with heat may assist in delivering a therapeutic species to a subject with a linker having a certain activation temperature. Similarly, combining focused ultrasound with an applied magnetic field may assist in delivering a therapeutic species to a subject with a linker having certain magnetic properties or covalently linked to a magnetic material such as a nanoparticle, microparticle, and the like.

In any of the embodiments disclosed here, a method 1000 of delivering a therapeutic species to a subject can include step 1010 of disposing a hydrogel and a therapeutic species in the subject, the hydrogel comprising a linker joining the hydrogel to the therapeutic species. Method 1000 can further include exposing 1020 the composition to pulsed waves of acoustic energy, thereby initiating a reversible retrograde cleavage reaction to severe the linker and decouple the therapeutic species from the hydrogel. Method 1000 can end after step 1020, or can further include various steps of encapsulating the therapeutic species within the hydrogel or coupling the therapeutic species with the hydrogel via a Diels-Alder reaction product comprising a first precursor on the hydrogel and a second precursor on the therapeutic species.

According to certain embodiments, a method 1100 for promoting controlled tissue regeneration in a subject is described. Method 1100 can include disposing 1110, against a tissue of the subject, a material comprising a Diels-Alder reaction product comprising a first precursor and a second precursor. Method 1100 can also include exposing 1120 the material to a first triggering event, thereby initiating a retro-Diels-Alder reaction of the material. Method 1100 may end after step 1120 or can optionally further include coupling 1130 a therapeutic species with the material via a Diels-Alder reaction product comprising a third precursor and a fourth precursor different than the first and second precursors. Method 1100 can further include exposing 1140 the therapeutic species to a second triggering event, thereby initiating a retro-Diels-Alder reaction of the third and fourth precursor to uncouple the therapeutic species. In addition, method 1100 can further include adjusting 1150 the second triggering event to comprise at least one of the waveform, pulse duration, or pulse repetition frequency different from the first triggering event such that the therapeutic species is uncoupled at a different rate than the retro-Diels-Alder reaction of the material.

The following examples further illustrate aspects of the present disclosure. However, they are in no way a limitation of the teachings or disclosure of the present disclosure as set forth herein.

### EXAMPLES

### Example 1—Materials

6-Maleimidohexanoic acid (90%), 2-furoic acid (98%), 2-thiophenecarboxylic acid (99%), methanol (anhydrous, 99.8%), chitosan (75-85% deacetylated, 50-190 kDa), glutaraldehyde (solution, 25% in H2O), hydrochloric acid (HCl, ACS Reagent, 37%), FITC-albumin (fluorescein isothiocyanate conjugate), N-hydroxysuccinimide (NHS, 98%), Proteinase K (from Tritirachium album, ≥30) units/mg protein), methanol-d4 (≥99.8 atom % D), and deuterium chloride solution (35 wt. % in D2, ≥99 atom % D) were purchased from Millipore Sigma (St Louis, MO). Tegaderm was acquired from 3M Health Care (St Paul, MN). HeLa cells were obtained from the Sartorius Cell Culture Facility of the Pennsylvania State University (University Park, PA). Fetal bovine serum (FBS) was acquired from Corning (Corning, NY). Dulbecco's Phosphate Buffered Saline (PBS) was obtained from Cytiva (Pittsburgh, PA). Disposable biopsy punches (Miltex, 4.0) mm diameter) were acquired from Integra LifeSciences (Princeton, NJ). Tissue culture plate inserts (polycarbonate membrane, translucent, 0.4 μm pore size) were bought from VWR (Radnor, PA). LIVE/DEAD Viability/Cytotoxicity kit, alamarBlue HS cell viability reagent, Quant-iT PicoGreen dsDNA assay kit, 1-ethyl-3-(3-dimethylaminopropyl) carbodiimide hydrochloride (EDC), Dulbecco's Modified Eagle Medium (DMEM), and antibiotic-antimycotic were purchased from Thermo Fisher Scientific (Waltham, MA). All reagents were used as received.

### Example 2—Example Synthesis of Diels-Alder Linkers

A variety of cycloadducts were used in the study disclosed herein. Furan DA (FDA) was the product of the Diels-Alder reaction between 2-furoic acid and 6-maleimidohexanoic acid. Thiophene DA (TDA) was the product between 2-thiophenecarboxylic acid and 6-maleimidohexanoic acid. The general cycloaddition reaction between a furan, thiophene, or pyrrole diene (Formula I) and 6-maleimidohexanoic acid is illustrated below. For the preparation of FDA, 1.75 g of 6-maleimidohexanoic acid and 0.93 g of 2-furoic acid were dissolved into 15 mL of methanol in a glass vial. The container was then sealed, protected from light using aluminum foil, and the reaction allowed to proceed for 7 days at room temperature under agitation. TDA was synthesized by combining 1.75 g of 6-maleimidohexanoic acid and 1.06 g of 2-thiophenecarboxylic with 15 mL of methanol. The glass vial used for the reaction was then sealed, protected from light, and heated at 60° C. in an oil bath for 3 days. Both cycloadducts were purified by column chromatography and characterized by 1H NMR spectroscopy and 13C NMR spectroscopy.

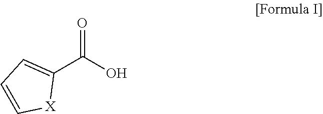

where X is S, O, or NH. Reacting Formula I with 6-(2,5-dioxo-2,5-dihydro-1H-pyrrol-1-yl) hexanoic acid generates Formula II Diels-Alder linker.

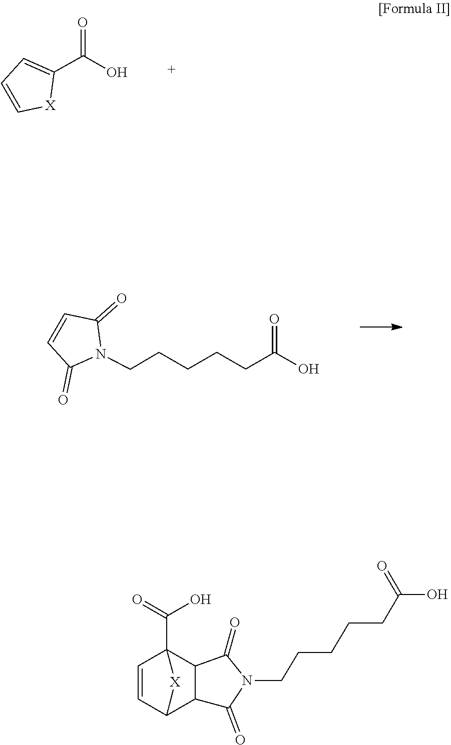

Chitosan (0.25 g) was dissolved in 5 mL of deionized water with 17 μL of 1 M HCl. EDC/NHS. 100 μL of a 100 μM solution were added and reacted with the chitosan for 15 min at room temperature. 500 μL of a Diels-Alder linker, such as, for example, FDA or TDA, were added to the mixture prior to casting it in a 35 mm diameter Petri dish. The hydrogels were left to crosslink overnight and were lyophilized before characterization.

### Example 3—Preparation of Chitosan Hydrogels

Chitosan (0.25 g) was dissolved in 5 mL of deionized water with 17 μL of 1 M HCl. EDC/NHS. 100 μL of a 100 μM solution were added and reacted with the chitosan for 15 min at room temperature. 500 μL of a Diels-Alder linker, such as, for example, FDA or TDA, were added to the mixture prior to casting it in a 35 mm diameter Petri dish. The hydrogels were left to crosslink overnight and were lyophilized before characterization. Control hydrogels without a thermally labile Diels-linker were obtained by crosslinking chitosan with 100 μL of a glutaraldehyde solution (100 μL of a 0.1% solution in water). For protein release studies, FITC-Albumin (100 μL of a stock solution at 0.5 mg/mL) was added to the chitosan prior to crosslinking. In some examples, chitosan was mixed with Formula II to form Formula III.

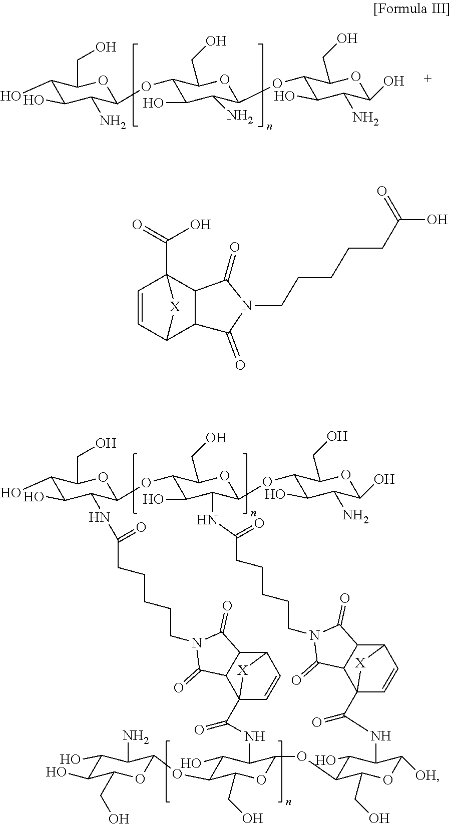

where X is S, O, or NH and n is an integer from 10 to 2200.

### Example 4—Characterization of Hydrogels

1H and 13C NMR spectra of the crosslinked hydrogels were collected at 298 K on a Bruker Avance-III-HD-500 (Bruker, Billerica, MA). FTIR spectra were recorded on a Bruker Vertex 70 in the 4000 to 550 cm⁻¹ range. Differential scanning calorimetry (DSC) analyses were carried out using a DSC Q2000 calorimeter (TA Instruments, New Castle, DE). Thermal properties were recorded between 25° C. and 200° C. at a heating rate of 10° C./min to determine the enthalpy of reaction. Nitrogen was used as purge gas.

### Example 5—PCL-Furan Synthesis

PCL Diol (3.0 g, 1.5 mmol) was reacted with MDI (Methylene diphenyl diisocyanate, 0.75 g, 3 mmol) using DMF (25mL) as a solvent in a 100 mL round bottom flask, equipped with a magnetic stir bar. The reaction was carried out at 60° C. under a nitrogen atmosphere for three hours. The solution was then cooled to room temperature. In the second step, furfuryl amine (0.133 mL, 1.5 mmol) was added dropwise into the isocyanate end-capped pre-polymer solution. Half an hour after adding the furfuryl amine, the temperature was increased to 70° C. and kept for 10 hours. After cooling to room temperature, a drop of furfuryl amine was added to the solution to ensure the isocyanate was all consumed. Half an hour later the whole solution was poured into excess diethyl ether to purify PCL Furan. After filtering and drying under vacuum at room temperature, the semi-dry product was placed in evaporating dishes and left at 37° C. overnight. The white powder polymer was obtained as the intermediate product PCL Furan. Control polymers without a thermally labile Diels-linker were obtained by crosslinking PCL cyanate (3 g) with isosorbide (0.147 g, 1 mmol). For degradation or protein release studies, FITC-Albumin (500 μL of a stock solution at 0.5 mg/mL) was added to the PCL prior to crosslinking. In some examples, formation of PCL linked with Diels-Alder linker is shown in the schematic below to generate Formula IV.

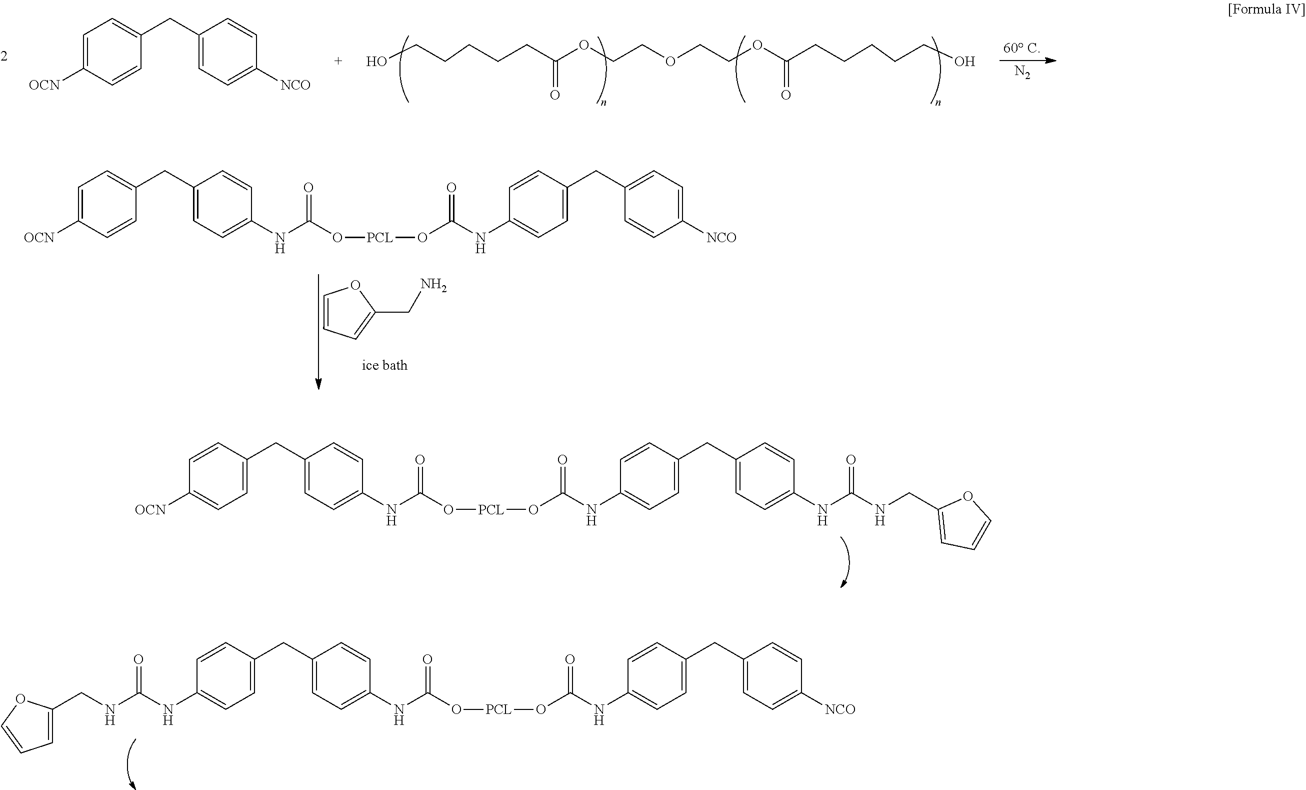

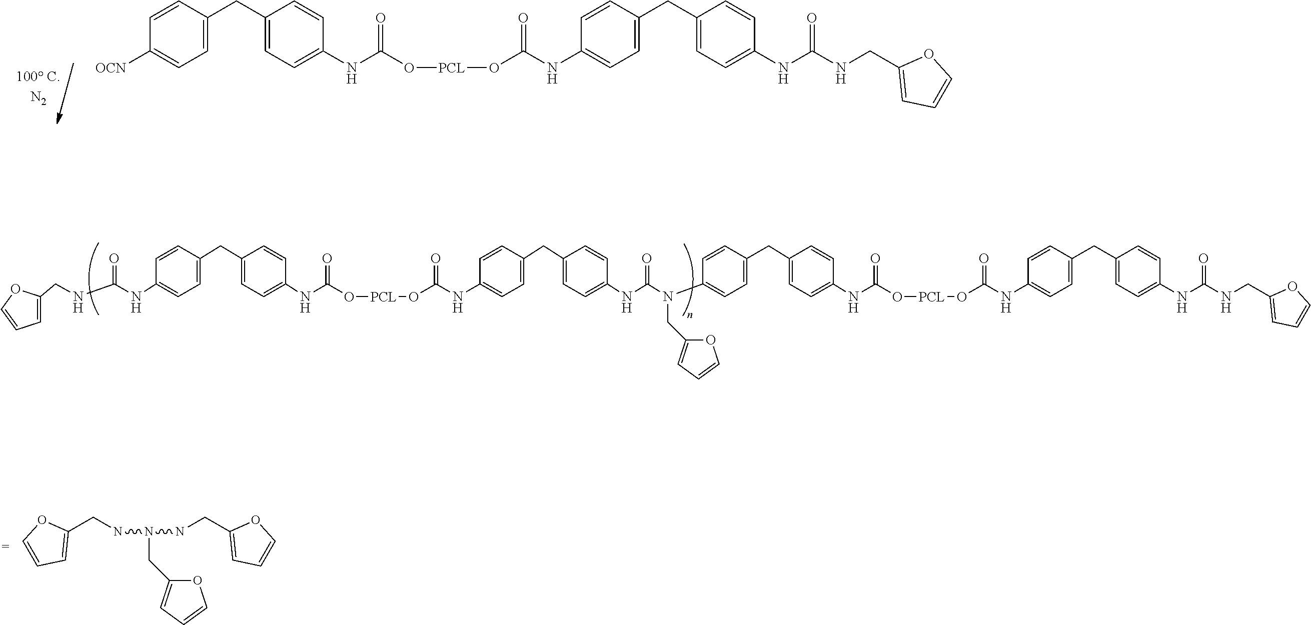

### Example 6—PCL-Furan Crosslinking with Linker

Bismaleimide (BMI) (0.39 g, 1 mmol) was dissolved in DMF (2 mL) then added to a solution of PCL Furan (3 g) dissolved in DMF (12.5 mL). A magnetic stir bar was placed in the 25 mL flask and the temperature of the water bath was set to be 50° C. The whole solution becomes a gel in about 5 hours after adding BMI. Four hours after adding BMI, FITC-Albumin (0.5 mL, 0.5 mg/mL) was added and allowed to stir for 10 minutes. The 15 mL solution was poured into a 100 mm diameter PTFE evaporating dish to avoid gelling in the flask The PTFE dish was put in a vacuum oven at 50° C. overnight. The cross-linking via DA reaction will further proceed to obtain a polymer film with a higher cross-linking density. Meanwhile, the solvent DMF was evaporated, and a flat cross-linked PCL-FDA polymer was obtained as the final product. Combining Formula IV with bismaleimide under nitrogen at 60° C. to initiate crosslinking, the PCL-linker structure in Formula V is formed, as depicted below.

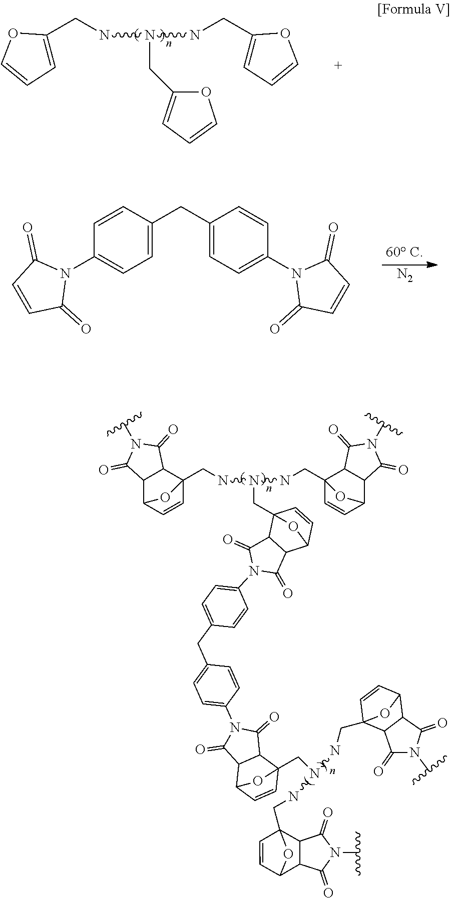

FTIR absorbance spectra of PCL diol, PCL cyanate, PCL-Furan (Formula IV) and PCL-FDA (Formula V) are provided in FIG. 4C. PCL Furan has high concentration of C═O causing broad peak at 1665 cm⁻¹. Between the diol and the two crosslinked samples are aromatic rings at 1650-1580 cm⁻¹ and 1550-1400 cm⁻¹ range. The small bumps at 3350 cm⁻¹ are amide N—H stretches. A lot of the broader peaks between 1250 cm⁻¹ and 1020 cm⁻¹ are due to C—N bonds. Stretches between 1342-1266 cm⁻¹ are associated with aromatic amines and 1310-1250 cm⁻¹ for aromatic esters (slightly more broad). FTIR absorbance spectra of PCL diol, PCL cyanate, thiophene methyl and PCL-TDAM are provided in FIG. 4D. FTIR absorbance spectra of PCL diol, PCL cyanate, thiophene ethyl and PCL-TDAE are provided in FIG. 4E. FTIR absorbance spectra of PCL diol, PCL CYA, PCL-ISO are provided in FIG. 4F. FTIR absorbance spectra of PCL-FDA, PCL TDAM, TCL TDAE, and PCL ISO are provided in FIG. 4G.

### Example 7—Biocompatibility

HeLa cells were cultured in Dulbecco's Modified Eagle Medium (DMEM) supplemented with 10% fetal bovine serum and 1% antibiotic-antimycotic. Cell culture flasks were kept in a humidified incubator with 5% CO₂ and a temperature of 37° C.

HeLa cells were seeded in 24-well plates at a density of 0.05×106 cells per well. Hydrogels were sectioned using disposable biopsy punches to obtain samples with a uniform geometry and volume. Hydrogels samples were added to the different wells using tissue culture plate inserts. Cell viability and proliferation assays were performed at day 1, day 3, and day 7 after exposing the cells to the hydrogels (see FIG. 7A). The metabolic activity of the cells was measured using an alamarBlue assay according to the manufacturer's recommendations. Cells were incubated at 37° C. for 3 hours with a 10% alamarBlue solution. The fluorescence intensity was then measured in each well at 560/590 nm (Excitation/Emission) using a Molecular Devices Spectramax M5 Microplate/Cuvette Reader. Cell viability was assessed using a LIVE/DEAD Viability/Cytotoxicity according to the manufacturer's protocol. Cells were washed with PBS and incubated at 37° C. for 30 min with a 2 μM calcein AM and 4 μM EthD-I working solution. Cells were then imaged with an Olympus IX73 fluorescence microscope (Olympus, Center Valley, PA). ImageJ (NIH, Bethesda, MD) was used for image processing. Total DNA content was used to determine the cell count. Proteinase K at a concentration of 0.5 mg/mL was added to the wells and plates incubated overnight at 56° C. to lyse the cells and release their DNA content. A PicoGreen dsDNA assay kit was used according to the manufacturer's recommendations to quantify the amount of dsDNA per sample. Equal volumes of PicoGreen dsDNA reagent were combined with the volumes of the wells and the fluorescence intensity was then measured in each well at 480/520 nm (Excitation/Emission) using a Molecular Devices Spectramax M5Microplate/Cuvette Reader.

To evaluate the cytocompatibility of the chitosan hydrogels crosslinked with Diels-Alder linkers, HeLa cells were exposed directly to hydrogels and their decomposition products. The metabolic activity, viability, and total cell numbers were measured after 1 day, 3 days, and 7 days (see FIGS. 8A and 8B). The hydrogels did not appear to induce any cytotoxicity in these direct exposure in vitro studies. No visible differences were present between the different groups on the LIVE/DEAD fluorescent images. Similarly, no significant difference could be measured between the Ch, Ch-FDA, and Ch-TDA groups for the metabolic activity and cell numbers over time. These results are in agreement with the literature, where chitosan has been described as a biomaterial with an excellent biocompatibility supporting robust cell proliferation. Likewise, other hydrogels crosslinked with Diels-Alder linkers have also been reported as being suitable for cell culture.

### Example 8—Immersion heating

Hydrogels containing FITC-Albumin were sectioned using 4 mm diameter disposable biopsy punches to obtain samples with a uniform geometry and volume. Each hydrogel sample was placed in a sealed microcentrifuge tube with 1 mL of PBS. Microcentrifuges tubes were then heated at either 40° C., 60° C., or 80° C. for 1 hour, 2 hours, or 6 hours. Water baths were used for temperatures of 40° C. and 60° C. but an oil bath was used for the 80° C. temperature to mitigate water evaporation. After immersion heating, samples were centrifuged (10 min, 1200× g), and three 150 μL aliquots of the supernatant were pipetted per sample into a 96-well plate. To evaluate the amount of FITC-albumin released by the retro Diels-Alder reaction (see FIG. 5A) the fluorescence intensity was measured in each well at 495/520 nm (Excitation/Emission) using a Molecular Devices Spectramax M5 Microplate/Cuvette Reader.

Thermal release immersion studies were performed with chitosan hydrogels crosslinked with either FDA or TDA linkers that contain FITC-albumin as a model protein. The amount of FITC-albumin released by the retro Diels-Alder reaction was determined via fluorescence intensity measurements. A higher protein release rate was measured for the FDA linker compared to the TDA linker with the same thermal treatments. Comparable Diels-Alder linkers replacing carboxyl groups for sulfhydryl groups on the dienes have similar protein release rates when releasing the payload from metal nanoparticles under thermal treatment. The experimental results showed that the furan-based Diels-Alder cycloadduct had a higher release rate than the thiophene-based cycloadduct, in correlation with the predicted energy barriers by density functional theory.

### Example 9—Ultrasound-mediated protein release

The synthesis and characterization of different Diels-Alder hydrogels have been previously described, but the interaction with focused ultrasound stimulation, hydrogel reorganization, and resulting controlled drug release has not previously been explored. The results described herein indicate the capability for focused ultrasound stimulation to drive the retro Diels-Alder reaction resulting in controlled release of entrapped protein payloads in Diels-Alder crosslinked hydrogels while allowing for real-time visualization of the ongoing process (see FIG. 6A). Increasing the focused ultrasound stimulation correlated with an increased rate of protein release indicating stimuli responsive control. The protein release rate for Ch-FDA was higher than observed for Ch-TDA, in agreement with the results from the thermal immersion study. These data indicate that the retro Diels-Alder reaction energy barriers correlate with the protein release rates, providing a means to tune the hydrogel stability upon exposure to focused ultrasound stimulation, through manipulation of the diene and dienophile composition.

### Example 10—Statistical Analysis

Data were analyzed using the GraphPad Prism 8 software. Results were expressed as mean±standard deviation (SD). Sample size (n) is indicated in the figure legends. Statistical analysis was performed via 2-way ANOVA with Tukey's post hoc testing. Statistical significance was set at p<0.05.

Example 11—Synthesis and Characterization of Diels-Alder Based Hydrogels

Linear heterobifunctional PEG derivatives containing acryloyl groups for crosslinking and amino groups for conjugation with dienes (TDAI, PDA, FDA, TDA2) were used to prepare PEG hydrogels as shown in FIG. 3C. Amide bonds were used to conjugate miR-210) and miR-148b to the dienophile (6-maleimidohexanoic acid) via an N-hydroxysuccinimide with amine reaction. miR-210-maleimide and miR-148b-maleimide were conjugated to the dienes on the PEG via Diels-Alder reaction. PEG at a molecular weight of 10 kDa at 15% (w/v) were used for rapid gelation. As provided in FIGS. 4A-4K, NMR and ESI-MS can characterize the reaction products, while DSC-TGA, FTIR, and rheology can characterize the hydrogels. The mechanical properties PEG-DA gels can be adjusted if needed by modifying the concentration, branching factor, or molecular weight of the macromonomers.

Density functional theory (DFT) models were used to provide essential quantum mechanical computations of forward and reverse barriers and energies of reactions for these 4 different DA cycloadduct linkers. Based on the modeled Gibbs free energy & enthalpy reaction barriers, 4,5-Dimethyl-N-(2-sulfanylethyl)-2-thiophenecarboxamide (TDA-1), pyrrole-2-carboxylic acid (PDA), 2-furanmethanethiol (FDA), 2-Thiophenemethanethiol (TDA-2) diene based DA cycloadduct linkers are expected to provide significantly different payload release kinetics profiles as provided in FIG. 9. See also Abu-Laban M, Kumal R R, Casey J, Becca J, LaMaster D, Pacheco C N, Sykes D G, Jensen L, Haber L H, Hayes D J. Comparison of thermally actuated retro-diels-alder release groups for nanoparticle based nucleic acid delivery. J Colloid Interface Sci. 2018:526:312-21. Epub 2018 May 12. doi: 10.1016/j.jcis.2018.04.085. PubMed PMID: 29751265: PMCID: PMC5994202 and Arrizabalaga J H, Casey J S , Becca J C, Jensen L, Hayes D J. Comparison of thermoresponsive Diels-Alder linkers for the release of payloads from magnetic nanoparticles via hysteretic heating. JCIS Open. 2021:4:100034. doi: https://doi.org/10.1016/j.jciso.2021.100034.

The reversible DA moieties described herein were used to link miRNAs into a hydrogel as shown in FIG. 3C. Hydrogels such as chitosan or PEG can be crosslinked using Diels-Alder linkers to form a therapeutic-releasing composition. Although FIG. 4A provides for hydrogel-FDA (2-furanmethanethiol) and hydrogel-TDA-1 (2-thiophenemethanethiol), additional linkers are contemplated. FIGS. 4B and 4L provide DSC-TGA and rheology, respectively, of hydrogel-FDA and hydroge-TDA-1 that demonstrate predictable differences in thermal and mechanical behavior correlating with the calculated reaction barriers provided in FIG. 9.

### Example 12—Focused Ultrasound Mediated Payload Release

FIG. 6A provides images indicating the capability for fUS stimulation to control the release of entrapped BSA protein payloads in Diels-Alder crosslinked hydrogels while allowing for real-time visualization of the ongoing process. Increasing fUS stimulation correlates with increased rate of protein release indicating stimuli responsive control, as shown in FIG. 5A. Based on the density functional theory (DFT) modeled reaction barriers, TDA-1, PDA, FDA, and TDA-2 Diels-Alder cycloadduct linkers can provide significantly different payload release kinetics. FIG. 7B shows HeLa cells cultured with FDA-Chitosan and TDA-Chitosan, after 3 days did not experience any cytotoxicity in vitro by use of the hydrogels.

### Example 13—Combinatorial Delivery of miR-210 and miR-148b Mimics in ADSCs Spheroids

To explore simultaneous osteogenic and endotheliogenic codifferentiation of stem cells, transfected ADSCs were combined into 3D spheroid cultures Transfection of miR-210 mimic resulted in upregulation of VE-cadherin and minimal RunX2 (FIG. 7B) expression compared to untreated controls. When spheroids were transfected with miR-148b alone, RUNX2 was upregulated with minimal VE-cadherin expression. Combining miR-148b and miR-210 resulted in greater RUNX2 and VE-cadherin expression compared to controls. This codifferentiation condition with transfected ASC spheroids closely represents the coordinated differentiation environment that progenitors encounter in vivo during bone regeneration.

Spatiotemporally controlled miR-210 mimic and miR-148b mimic delivery by Diels-Alder crosslinked polyethylene glycol (PEG) hydrogels modulates bone formation both in vitro and in vivo. The retro Diels-Alder reaction kinetics correlates with miRNA mimics release rates when stimulated by fUS. In addition, the release of miR-210 and miR-148b mimics can be controlled sequentially by tuning the fUS energy to the DA retro reaction barrier.

As shown in FIG. 6A, fUS stimulation is capable of controlling the release of entrapped BSA protein payloads in Diels-Alder crosslinked hydrogels while allowing for real-time visualization of the ongoing process. Increasing fUS stimulation correlates with increased rate of protein release indicating stimuli responsive control (FIG. 5A). The fUS response and associated release of miR-210 and miR-148b mimic from the hydrogel have been explored. In particular, the critical parameters for the fUS stimulation, as well as the impact of miR-210 and miR-148b mimic concentration and the DA moiety composition in vitro.

TABLE 1
In Vitro release and cytocompatibility

| Nucleic | Diels-Alder | Dosages | Focused Ultrasound | | |
|---|---|---|---|---|---|
| Acids | Group | (nM) | Conditions | Assays | Criteria |
| miR-210 | TDA1- | 100-500 | Pressure: 78 MPa(+) | Picogreen (n = 5) | >95% Viability |
| mimic | maleimide | | and 21 MPa(−) | Alamar Blue (n = 5) | for 28 days |
| | PDA- | | Pulse Length: 0.1- | Mass Loss (n = 10) | <25% loss of |
| | maleimide | | 100 ms at 0.1-100 Hz | | 28 days |
| miR-148b | FDA- | | Time: 1-5 min | Rheology (n = 10) | \|G*\| = 20 kPa +/− |
| mimic | maleimide | | | | 5 kPa |
| | TDA2- | | | Release (n = 5) | <10% spurious |
| | maleimide | | | | nucleic acid |
| | | | | | release |

The influence of DA linker composition on the fUS response of PEG hydrogels and the associated release of miR-210 and miR-148b mimics has been assessed as outlined in Table 1. PEG hydrogels were prepared with FDA, PDA, TDA-1 and TDA-2 linkers and the release kinetics were determined for miR-210 and miR-148b mimics as a function of fUS conditions using fluorescence-based retention assays. Cell proliferation and viability were assessed by DNA quantification and metabolic assays.

### Example 14—Focused Ultrasound Stimulation

For the fUS stimulation, a Sonic Concepts High Intensity Focused Ultrasound transducer (64-mm, 1.2-MHz, f#=1 with 3.6 MHz third Harmonic) was utilized. The peak pressure amplitude ranges from up to 78 MPa positive and 21 MPa negative and was applied with pulse lengths of 0.1-100 ms repeated at 0.1-100 Hz for a total application time of 1 to 5 min.

### Example 15—Release of miRNA Mimics

miR-210 and miR-148b mimics were labeled with Cy3 and FAM respectively. As shown in FIG. 3C, 200 nM miR-210 or miR-148b were bound in each Diels-Alder-PEG hydrogel. Cy3 labelled miR-210-maleimide or FAM miR-148b-maleimide were coupled to the PEG-diene. The release kinetics from the hydrogels were determined for Diels-Alder linked miR-210 and miR-148b mimic as a function of fUS conditions using fluorescence-based retention assays. Mass loss after fUS stimulation gels were measured. Temperature of the gel during fUS were monitored with a data logger thermocouple and a near IR camera.

The miRNA mimic release scales with fUS energy delivery and optimal fUS conditions can be expected in the range of 300 mV amplitude (peak pressures of 68 MPa positive, 15 MPa negative) with 10-ms pulses delivered at 1 Hz for up to 5 min. miR-210 and miR-148b mimic release were punctuated and significant release allowed to occur up to 4 hrs after fUS stimulation. The kinetics of miRNA mimics release followed the trend: TDA1>PDA>FDA>TDA2 in DA hydrogels. A shear modulus of ˜20 kPa is expected. The hydrogels did not induce significant cytotoxicity in MSC in vitro. Animals experienced no significant host response or side effects from the hydrogels or fUS stimulation.

### Example 16—Cytocompatibility Evaluation

The cytocompatibility of the ultrasound disrupted hydrogel was evaluated in a transwell MSC culture model. CD34+ EPC and MSC were characterized and cultured according to standard protocols. Cells from four donors were seeded at 200,000 cells per cm³ on transwell inserts and placed in wells with Diels-Alder-PEG hydrogel, fUS activated and cultured for a period of up to 4 weeks. The proliferation, viability and the markers of stemness (Nanog and Sox2) were assessed weekly by DNA content, Alamar Blue assay and RT-PCR according to standard methods.

A limited scope in vivo femoral defect implantation was conducted to determine basic pharmacokinetics, in vivo degradation rates and biocompatibility according to Table 2 (below). This surgical procedure, established at PSU under IACUC protocol PROTO201800516 was conducted according to a modification of standard methods. Briefly, 8-10 week old Sprague-Dawley rats (250-300 g) were anesthetized using 100-120 mg/kg Ketamine, 10-16 mg/kg xylazine. 6 mm osseous critical size defects were created in the right femoral diaphysis of adult rats. A predrilled, high-density polyethylene fixation plate (4×4×23 mm) was then be placed to maintain osseous stability. A collagen sponge and a 6 mm×2 mm cylindrical PEG hydrogel with Diels-Alder linker containing 200 nM Cy3 labeled miR-210 mimic and 200 nM FAM labeled miR-148b mimic was inserted into the surgical site to fill the defect and was sutured to the periosteum proximally and distally. A collagen sponge with concentration matched miR-210 and miR-148b mimics served as the control. The surgical site was closed in layers and the animal was monitored per established post-operative animal care protocols. At 3, 7 and 14 days post-surgery, fUS was applied to the defect site of half of the animals, while half received no fUS (control). As described supra, a Sonic Concepts High Intensity Focused Ultrasound transducer (64-mm, 1.2-MHz, f#=1 with 3.6 MHz third Harmonic) was utilized for the fUS stimulation. At each time point, animals were euthanized with serum and tissue was collected. Fluorescence microscopy was used to examine Cy3 and FAM labeled miRNA mimics distribution in the surgical site. miR-210 and miR-148b mimic release were determined by comparing remaining miRNA mimic fluorescence in the gel from fUS groups and control. Degradation was analyzed at each time point by histology. PEG ELISA assay (Abcam) was conducted to detect PEG degradation products in serum. To determine biocompatibility, surgical site inflammation and weight were observed every other day. At day 14, surgical site tissue, liver, spleen, kidney, brain and cardiac tissue were extracted for histology and enzyme markers, serum alanine aminotransferase (ALT), aspartate aminotransferase (AST), alkaline phosphatase (ALP) levels were measured. Based on previous experiments described supra, four (n=4) rats were required for each group. This study required a total of 96 animals: two groups (fUS and no fUS), four hydrogel compositions (TDA-1, PDA, FDA, TDA-2) at 3 time points (4 rats×2groups×4 compositions×3 time points).

TABLE 2
Experimental Matrix N = 4 rates per group (2 male/2 female)

| | | Focused Ultrasound | |
|---|---|---|---|
| Nucleic Acids | Diels-Alder Group | Time Points | Assays |
| miR-210 | TDA1-maleimide | 3, 7, and | In vivo fluorescent imaging miR- |
| mimic | | 14 days | 210 & miR-148b mimics |
| | PDA-maleimide | | ELISA-PEG |
| miR-148b | FDA-maleimide | | Organ Histology |
| mimic | TDA2-maleimide | | Hydrogel Degradation |

### Example 17—Exploration of fUS-Mediated Release of miR-210 and miR-148b Mimics from Hydrogels on Bone Regeneration in Segmental Defect Repair

Compared to miR-148b mimic alone, miR-210 and miR-148b mimic co-induction in MSC significantly upregulated osteogenic and endotheliogenic marker expression, but the timing of induction influenced expression. Therefore, the sequential controlled delivery of miR-210 and miR-148b using a fUS mediated system will result in increased endotheliogenic and osteogenic differentiation, resulting in improved segmental defect closure in a fUS dependent manner. Based on Gibbs free energy & enthalpy reaction barriers the four Diels-Alder linkers will result in significantly different release kinetics (FIG. 9).

To demonstrate controlled release of miR-210 mimic and miR-148b mimic and bone regeneration, a femoral defect model was conducted. fUS was applied to the defect sites at 3, 7, and 14 days post-surgery to mimic the differentiation timeline described supra. Vascularization and bone regeneration were observed at 3, 6 and 12 weeks to capture the influence of serial delivery on bone production and remodeling.

### Example 18—In Vitro Evaluation of fUS-Mediated miR-148b Release on Osteogenesis in MSCs

The activity of fUS released miR-210 and miR-148b mimics in the endotheliogenic and osteogenic induction of CD34+EPC and MSC was assessed by serial fUS stimulation of PEG-DA gels using transwell culture model activated at 3, 7 and 14 days, as outlined in Table 3. The impact of thermalization on miR-210 and miR-148b mimic activity was assessed in these experiments by comparing activity of fUS released miRNA mimic to control untreated miRNA mimic. Hydrogels were prepared and transwell MSC cell culture were also conducted. Progenitor cell differentiation was assessed by Raman Spectroscopy, qPCR, immunohistochemistry and colorimetric stains weekly for 28 days. The expression of endotheliogenic markers: CD31, CD34, and vWF and osteogenic regulators, NOG, RUNX1 and osteogenic markers: ALP, RunX2, OP, and OCN were assessed weekly by qRT-PCR, ELISA, immunofluorescence and colorimetric stains. Mineralization was assessed by Alizarin Red Stain and OsteoImage served as end point measures of in vitro osteogenesis.

TABLE 3
In Vitro evaluation of fUS-mediated differentiation

| Nucleic | Diels-Alder | Dosages | Focused Ultrasound | | |
|---|---|---|---|---|---|
| Acids | Group | (nM) | Conditions | Assays | Criteria |
| miR-210 | TDA1- | 100-500 | Pressure: 78 MPa(+) | Picogreen (n = 5) | >95% Viability for 28 |
| mimic | maleimide | | and 21 MPa(−) | Alamar Blue | days |
| | | | Pulse Length: 0.1- | (n = 5) | |
| | PDA- | | 100 ms at 0.1-100 Hz | Mineralization | >10 fold greater than |
| | maleimide | | Time: 1-5 min | (osteoimage) | control |
| | | | | (n = 5) | |
| miR-148b | FDA- | | | vWF, CD31, | >5 fold upregulation |
| mimic | maleimide | | | CD34, OCN, | compared to control |
| | | | | OPN (PCR and | |
| | | | | ELISA) | |
| | TDA2- | | | ALP (PCR and | >3 fold upregulation |
| | maleimide | | | colorimetric) | compared to control |

### Example 19—Bone Regeneration in vivo

The influence of spatiotemporally modulated delivery of miR-210 and miR-148b mimics on the closure of critical sized femoral defects were evaluated. The in vivo study was conducted using the two Diels-Alder compositions, although additional Diels-Alder linkers are expected to work comparatively to the two compositions tested. The Diels-Alder linker with lowest energy retro reaction barrier with demonstrated in vivo functionality and biocompatibility was used to link miR-210 to hydrogel. The Diels-Alder linker with the highest energy retro reaction barrier tolerated was used for miR-148b. This provided largest difference in fUS release rates between miR-210 and miR-148b mimics. Defects were created according to the method described supra. Collagen sponge was press fit into the defect and Diels-Alder PEG hydrogels with miR-210 and miR-148b mimics were placed next to the critical sized femoral defects. Collagen alone did not result in critical sized defect femoral defect closure. A collagen sponge with miR-210 and miR-148b and fUS served as the control. Serial fUS treatments at 3, 7 and 14 days post-surgery were applied to the defect site to release miR-210 mimic (Day 3) and miR-148b mimic (Day 7 and 14) locally to the defect. The study included fUS and no fUS groups and a collagen miR-210 and miR-148b mimic (control) group. At 3, 6 and 12 weeks post-surgery animals were euthanized. Bone formation were assessed by Raman spectroscopy, histology and μCT. Tissues underwent histological (analine blue, pentachrome staining, immunostaining for CD31, CD34, Runx2 and OCN), Osteoimage and μCT to assess mineralization, bone formation and vascularization in tissue near the defect site. Heterotopic ossification (HO) was assessed in the whole animal. Similarly, μCT was performed for imaging vasculature using micofil to increase contrast. The scans were registered in 3D space with Avizo software using landmark-based and intensity-based approaches. Vessel density, vessel separation and inter-connectivity were determined using Avizo software. Tissue samples from the defect were digested and lysate tested against the 41 target Human Bone Metabolism Antibody Array B (Abcam). Sample sizes of eight rats/group allowed detection of approximately ˜20% pairwise difference in bone volume (A=20%) as measured by μCT between experimental/control groups, assuming 80% power and alpha =0.0115. A total of 96 animals were used (8 rats×2 groups×2 compositions (Exp and control)×3 time points).

Serial delivery of miR-210 and miR-148b mimics increased expression of endotheliogenic and osteogenic markers for the progenitors in vitro. Serial miR-210 and miR-148b mimic delivery in vivo improved vascularization of the defect and defect closure compared to both no fUS and collagen gel control cohorts. Animals receiving serial fUS stimulated hydrogels experienced improved bone volume compared to no fUS groups and collagen hydrogel group.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A composition comprising:
a hydrogel comprising a linker comprising a Diels-Alder cyclo-addition reaction product; and
a therapeutic species coupled to the hydrogel,
wherein the linker is configured to undergo a reversible retrograde cleavage reaction to release the therapeutic species from the hydrogel upon exposure to a triggering event.

2. The composition of claim 1, wherein the therapeutic species is encapsulated within the hydrogel.

3. The composition of claim 1, wherein the triggering event comprises pulsed waves of acoustic energy.

4. The composition of claim 3, wherein the pulsed waves of acoustic energy comprises a waveform, a pulse duration, and a pulse repetition frequency.

5. The composition of claim 4,
wherein the waveform comprises a positive peak pressure amplitude ranging from about 40 megapascals (MPa) to about 100 MPa and a negative peak pressure amplitude ranging from about 10 MPa to about 30 MPa;
wherein the pulse duration comprises a number of cycles ranging from about 1 cycle to about 20,000 cycles within a timeframe ranging from about 1 microseconds (μs) to about 20 milliseconds (ms);
wherein the pulse repetition frequency comprises a frequency ranging from about 0.1 hertz (Hz) to about 100 Hz; and
wherein a period of treatment time ranges from about 30 seconds to about 300 seconds.

6. The composition of claim 1, wherein the hydrogel comprises a biocompatible polymer.

7. The composition of claim 6, wherein the biocompatible polymer comprises substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxy butyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

8. The composition of claim 1, wherein the therapeutic species comprises at least one of a small molecule, a nucleic acid, a peptide, a protein, a microRNA mimetic, and combinations thereof.

9. The composition of claim 1, wherein the linker comprises a reaction product of a diene and a dienophile.

10. The composition of claim 9, wherein the diene comprises at least one of a substituted or unsubstituted furan, thiophene, or pyrrole.

11. The composition of claim 9, wherein the dienophile comprises at least one of a substituted or unsubstituted alkene or alkyne.

12. A composition comprising:
a first material comprising a first precursor; and
a first therapeutic species comprising a second precursor,
the first and second precursor forming Diels-Alder cyclo-addition reaction product configured to undergo a retro-Diels-Alder reaction upon exposure to a first triggering event to release the first therapeutic species.

13. The composition of claim 12, further comprising:
a second material comprising a third precursor; and
a second therapeutic species comprising a fourth precursor,
the third and fourth precursor forming a Diels-Alder cyclo-addition reaction product, the third precursor and fourth precursor different than the first precursor and the second precursor.

14. The composition of claim 13, wherein the third and fourth precursors are configured to undergo a retro-Diels-Alder reaction upon exposure to a second triggering event to release the second therapeutic species.

15. The composition of claim 14, wherein the first triggering event is different from the second triggering event, such that the first therapeutic species and second therapeutic species are released at different triggering events.

16. The composition of claim 15, wherein at least one of the first triggering event or the second triggering event comprises pulsed waves of acoustic energy.

17. The composition of claim 16, wherein the pulsed waves of acoustic energy comprises a waveform, a pulse duration, and a pulse repetition frequency.

18. The composition of claim 17, wherein when the first triggering event and the second triggering event comprise pulsed waves of acoustic energy, the second triggering event comprises pulsed waves of acoustic energy comprising at least one of the waveform, pulse duration, or pulse repetition frequency different from the first triggering event.

19. The composition of claim 17,
wherein the waveform comprises a positive peak pressure amplitude ranging from about 40 megapascals (MPa) to about 100 MPa and a negative peak pressure amplitude ranging from about 10 MPa to about 30 MPa;
wherein the pulse duration comprises a number of cycles ranging from about 1 cycle to about 20,000 cycles within a timeframe ranging from about 1 microseconds (μs) to about 20 milliseconds (ms);
wherein the pulse repetition frequency comprises a frequency ranging from about 0.1 hertz (Hz) to about 100 Hz; and
wherein a period of treatment time ranges from about 30 seconds to about 300 seconds.

20. The composition of claim 13, wherein the first material comprises a biocompatible polymer.

21. The composition of claim 20, wherein the second material comprises a biocompatible polymer.

22. The composition of claim 21, wherein the biocompatible polymer comprises substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxy butyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

23. The composition of claim 12, wherein the first therapeutic species comprises at least one of a small molecule, a nucleic acid, a peptide, a protein, a microRNA mimetic, and combinations thereof.

24. The composition of claim 12, wherein the first precursor comprises a diene selected from a substituted or unsubstituted furan, thiophene, or pyrrole.

25. The composition of claim 12, wherein the second precursor comprises a dienophile comprising a substituted or unsubstituted alkene or alkyne.

26. The composition of claim 13, wherein the third precursor comprises a diene selected from a substituted or unsubstituted furan, thiophene, or pyrrole.

27. The composition of claim 13, wherein the fourth precursor comprises a dienophile comprising a substituted or unsubstituted alkene or alkyne.

28. A method of delivering a therapeutic species to a subject, the method comprising:
disposing a composition comprising a hydrogel and a therapeutic species in the subject, the hydrogel comprising a linker joining the hydrogel to the therapeutic species; and
exposing the composition to pulsed waves of acoustic energy, thereby initiating a reversible retrograde cleavage reaction to severe the linker and decouple the therapeutic species from the hydrogel.

29. The method of claim 28, further comprising encapsulating the therapeutic species within the hydrogel.

30. The method of claim 28, further comprising coupling the therapeutic species with the hydrogel via a Diels-Alder reaction product comprising a first precursor on the hydrogel and a second precursor on the therapeutic species.

31. The method of claim 28, wherein the pulsed waves of acoustic energy comprises a waveform, a pulse duration, and a pulse repetition frequency.

32. The method of claim 31,
wherein the waveform comprises a positive peak pressure amplitude ranging from about 40 megapascals (MPa) to about 100 MPa and a negative peak pressure amplitude ranging from about 10 MPa to about 30 MPa;
wherein the pulse duration comprises a number of cycles ranging from about 1 cycle to about 20,000 cycles within a timeframe ranging from about 1 microseconds (μs) to about 20 milliseconds (ms);
wherein the pulse repetition frequency comprises a frequency ranging from about 0.1 hertz (Hz) to about 100 Hz; and
wherein a period of treatment time ranges from about 30 seconds to about 300 seconds.

33. The method of claim 28, wherein the hydrogel comprises a biocompatible polymer.

34. The method of claim 33, wherein the biocompatible polymer comprises substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxybutyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

35. The method of claim 28, wherein the therapeutic species comprises at least one of a small molecule, a nucleic acid, a peptide, a protein, a microRNA mimetic, and combinations thereof.

36. The method of claim 28, wherein the linker comprises a Diels-Alder reaction product comprising a diene precursor and a dienophile precursor.

37. The method of claim 36, wherein the diene precursor comprises at least one of a substituted or unsubstituted furan, thiophene, or pyrrole.

38. The method of claim 37, wherein the dienophile precursor comprises at least one of a substituted or unsubstituted alkene or alkyne.

39. A method of promoting controlled tissue regeneration in a subject, the method comprising:
disposing, against a tissue of the subject, a material comprising a Diels-Alder reaction product comprising a first precursor and a second precursor; and
exposing the material to a first triggering event, thereby initiating a retro-Diels-Alder reaction of the material.

40. The method of claim 39, further comprising encapsulating a therapeutic species within the material.

41. The method of claim 39, further comprising coupling a therapeutic species with the material via a Diels-Alder reaction product comprising a third precursor and a fourth precursor different than the first and second precursors.

42. The method of claim 41, further comprising exposing the therapeutic species to a second triggering event, thereby initiating a retro-Diels-Alder reaction of the third and fourth precursor to uncouple the therapeutic species.

43. The method of claim 42, wherein at least one of the first triggering event or the second triggering event comprises pulsed waves of acoustic energy.

44. The method of claim 43, wherein the pulsed waves of acoustic energy comprises a waveform, a pulse duration, and a pulse repetition frequency.

45. The method of claim 44, further comprising adjusting the second triggering event to comprise at least one of the waveform, pulse duration, or pulse repetition frequency different from the first triggering event such that the therapeutic species is uncoupled at a different rate than the retro-Diels-Alder reaction of the material.

46. The method of claim 44, further comprising adjusting the second triggering event to comprise at least one of the waveform, pulse duration, or pulse repetition frequency approximately identical to the first triggering event such that the therapeutic species is uncoupled at a similar rate as the retro-Diels-Alder reaction of the material.

47. The method of claim 44,
wherein the waveform comprises a positive peak pressure amplitude ranging from about 40 megapascals (MPa) to about 100 MPa and a negative peak pressure amplitude ranging from about 10 MPa to about 30 MPa;
wherein the pulse duration comprises a number of cycles ranging from about 1 cycle to about 20,000 cycles within a timeframe ranging from about 1 microseconds (μs) to about 20 milliseconds (ms);
wherein the pulse repetition frequency comprises a frequency ranging from about 0.1 hertz (Hz) to about 100 Hz; and
wherein a period of treatment time ranges from about 30 seconds to about 300 seconds.

48. The method of claim 39, wherein the material comprises a biocompatible polymer.

49. The method of claim 48, wherein the biocompatible polymer comprises substituted or unsubstituted polyurethane, polyethylene glycol, polycaprolactone, poly(methyl vinyl ether-alt-maleic acid), polylysine, polyglycolic acid, poly-L-lactic acid copolymers, polyhydroxybutyrate, hydroxyvalerate copolymers, poly-L-lactide, polydioxanone, polycarbonates, polyanhydrides, chitosan, chitin, cellulose, starch, glycogen, gelatin, amylose, hyaluronic acid, alginate, and combinations thereof.

50. The method of claim 41, wherein the therapeutic species comprises at least one of a small molecule, a nucleic acid, a peptide, a protein, a microRNA mimetic, and combinations thereof.

51. The method of claim 39, wherein the first precursor comprises a diene comprising a substituted or unsubstituted furan, thiophene, or pyrrole.

52. The method of claim 39, wherein the second precursor comprises a dienophile comprising a substituted or unsubstituted alkene or alkyne.
